(19) United States
(12) Patent Application Publication
(10) Pub. No.: US 2025/0304681 A1
(43) Pub. Date: Oct. 2, 2025

(54) ANTI-RAGE ANTIBODY, EXTRACELLULAR VESICLE, AND PREPARATION METHOD AND USE THEREOF

(71) Applicant: BEIJING ECHO BIOTECH CO., LTD., Beijing (CN)

(72) Inventors: Libo Zhao, Beijing (CN); Hang Zhao, Beijing (CN); Guanyi Kong, Beijing (CN); Jiaxun Zhang, Beijing (CN); Zhi Li, Beijing (CN)

(21) Appl. No.: 19/030,060

(22) Filed: Jan. 17, 2025

(30) Foreign Application Priority Data
Mar. 29, 2024 (CN) 2024103713076

(51) Int. Cl. C07K 16/28; A61K 47/68; A61K 47/69

(52) U.S. Cl. CPC C07K 16/2803; A61K 47/6849; A61K 47/6901; C07K 2317/32; C07K 2317/52; C07K 2317/622; C07K 2317/92

(57) ABSTRACT

The application discloses an anti-RAGE antibody, which can target RAGE antigen targets of different species such as human, murine and monkey. The application further discloses a conjugate, a bispecific antibody, a multispecific antibody, an immune cell, and a fusion protein which respectively comprises the anti-RAGE antibody, and further discloses a method for displaying the anti-RAGE antibody on extracellular vesicles. The anti-RAGE antibody provided by the application is applied to display on extracellular vesicles, endowing the extracellular vesicles with targeting ability, and finally enriching the extracellular vesicles in specific lesion tissues or organs with high expression of RAGE antigens, thereby facilitating the delivery, release and therapeutic effects of other drug molecules loaded on the extracellular vesicles in the specific lesion tissues.

### REFERENCE TO SEQUENCE LISTING SUBMITTED ELECTRONICALLY

The Sequence Listing, which is a part of the present disclosure, is submitted concurrently with the specification as an XML file. The name of the file containing the Sequence Listing is “70816_SeqListing.xml”, which was created on Dec. 31, 2024 and is 28,826 bytes in size. The entire contents of the Sequence Listing are incorporated herein by reference.

### TECHNICAL FIELD

The present application belongs to the field of bioengineering, and particularly relates to an anti-RAGE antibody, an engineered extracellular vesicle, and a preparation method and use thereof.

### BACKGROUND ART

The receptor for advanced glycation end products (RAGE, or AGER) is a multi-ligand cell surface member of the immunoglobulin superfamily. RAGE consists of an extracellular domain, a single transmembrane domain and a cytoplasmic tail. The extracellular domain of the receptor consists of one V-type immunoglobulin domain and two C-type immunoglobulin domains. RAGE is expressed by a variety types of cells, such as endothelial, smooth muscle cells, macrophages and lymphocytes in many different tissues, including lung, heart, kidney, skeletal muscle, and brain. RAGE is involved in many important pathological responses, including Alzheimer's disease, diabetes, inflammation and cancer. RAGE stimulates the growth, survival and metastasis of cancer. In addition, RAGE expression is closely associated with gastric cancer invasion and metastasis. Moreover, there are reports indicating that blocking RAGE can inhibit gastric cancer cell invasion. It has been reported that, Ser82 enhances ligand affinity and upregulates receptor signaling through mitogen-activated protein kinase (MAPK) and nuclear factor κB (NF-κB). RAGE interacts with advanced glycation end products (AGEs), S100 proteins, etc. The interaction between the ligand and the receptor RAGE activates important cellular pathways, including MAPK, Cdc42/Rac and NF-κB signaling pathways.

The vast majority of anti-RAGE antibody molecules reported so far have high affinity only for RAGE from a single species, but the single antigen species limits the versatility of the antibody. In addition, there are few applications for the modification of RAGE antibodies in the prior art. Currently, only a few research papers have reported EVs targeting RAGE antigens, but they used RAGE-binding peptides (the RAGE-binding domain truncated from the HMGB1 of RAGE ligand). There have been no reports of extracellular vesicles displaying RAGE antibody fragments.

### SUMMARY

In view of the above problems in the prior art, the present application provides an anti-RAGE antibody that can target RAGE antigen targets of different species such as human, murine and monkey, and has high application value both in scientific research and clinical application. The present application further provides a method for displaying the above anti-RAGE antibody on extracellular vesicles. The anti-RAGE antibody according to the present application is applied to display on the extracellular vesicles and endows targeting ability to the extracellular vesicles, thereby ultimately enriching the extracellular vesicles in specific lesion tissues or organs (such as the lungs) where the RAGE antigen is highly expressed, and facilitating the delivery, release and therapeutic effects of other drug molecules loaded on the extracellular vesicles in the specific lesion tissues.

The particular technical solutions of this application are as follows:

1. An anti-RAGE antibody, wherein the antibody comprises three heavy chain complementary determining regions (CDR-H) and three light chain complementary determining regions (CDR-L), the three heavy chain complementary determining regions are CDR-H1, CDR-H2 and CDR-H3, and the three light chain complementary determining regions are CDR-L1, CDR-L2 and CDR-L3, wherein
the CDR-H1 comprises the amino acid sequence represented by SEQ ID NO: 1;
the CDR-H2 comprises the amino acid sequence represented by SEQ ID NO: 2;
the CDR-H3 comprises the amino acid sequence represented by SEQ ID NO: 3;
the CDR-L1 comprises the amino acid sequence represented by SEQ ID NO: 4;
the CDR-L2 comprises the amino acid sequence represented by SEQ ID NO: 5; and
the CDR-L3 comprises the amino acid sequence represented by SEQ ID NO:6.

2. The anti-RAGE antibody according to item 1, comprising a human universal framework region.

3. The anti-RAGE antibody according to item 1 or 2, wherein the antibody comprises a heavy chain variable region and a light chain variable region, wherein
the heavy chain variable region comprises the amino acid sequence represented by SEQ ID NO:7, or an amino acid sequence having at least 90%, 91%, 92%, 93%, 94%, 95%, 96%, 97%, 98% or 99% identity with SEQ ID NO:7; and
the light chain variable region comprises the amino acid sequence represented by SEQ ID NO:8, or an amino acid sequence having at least 90%, 91%, 92%, 93%, 94%, 95%, 96%, 97%, 98%, or 99% identity with SEQ ID NO:8.

4. The anti-RAGE antibody according to any one of items 1-3, which is a human antibody, a humanized antibody or a chimeric antibody.

5. The anti-RAGE antibody according to any one of items 1-4, which is a full-length IgG antibody, preferably a full-length IgG1 antibody.

6. The anti-RAGE antibody according to any one of items 1-5, which is any one antigen-binding fragment selected from the group consisting of: Fv, Fab, F(ab′)2, Fab′, Fd, dsFv, scFv, sc(Fv)2, dAb, isolated complementary determining region, domain-specific antibody, single domain antibody, domain-deleted antibody, CDR-grafted antibody, diabody, triabody, tetrabody and minibody;
preferably, the anti-RAGE antibody binds to RAGE antigens derived from human, monkey and murine.

7. A conjugate comprising the anti-RAGE antibody according to any one of items 1-6.

8. The conjugate according to item 7, comprising a therapeutic agent conjugated to the anti-RAGE antibody;
preferably, the therapeutic agent is any one or more selected from the group consisting of: a cytotoxic agent, a hormone preparation, a small molecule targeting preparation, a proteasome inhibitor, an immunomodulator, an angiogenesis inhibitor, a cell proliferation inhibitor, a pro-apoptotic agent, a cytokine, and an activator of co-stimulatory molecule.

9. A bispecific or multispecific antibody comprising the anti-RAGE antibody according to any one of items 1-6.

10. An immune cell comprising the anti-RAGE antibody according to any one of items 1-6 or a nucleic acid encoding such an antibody.

11. The immune cell according to item 10, which is an NK cell, a T cell or a dendritic cell.

12. An antibody modified with lipophilic group, wherein the antibody is the anti-RAGE antibody according to any one of items 1-6, and preferably the lipophilic group is any one or two or more selected from the group consisting of: 1,2-distearoyl-sn-glycero-3-phosphoethanolamine (DSPE), dioleoylphosphatidylethanolamine (DOPE), and cholesterol.

13. A fusion protein comprising the anti-RAGE antibody according to any one of items 1-6 and one or more polypeptides or proteins.

14. The fusion protein according to item 13, wherein the polypeptide or protein is a scaffold protein or a truncation or variant thereof, an affinity pairing molecule, an antibody or a cytokine.

15. The fusion protein according to item 14, wherein the affinity pairing molecule is any one selected from the group consisting of: NbALFA/ALFA, monomer streptavidin/biotin, Strep-tag II/Strep-Tactin, N-terminal intein (Intein N)/C-terminal intein (Intein C), Spy Tag/Spy Catcher, and Protein A/Fc domain.

16. The fusion protein according to item 14, wherein the scaffold protein is a transmembrane protein;
preferably, the scaffold protein is any one or two or more selected from the group consisting of: PLXNA1, MFGE8, PTGFRN, BASP1 and PDGFR;
preferably, the antibody is any one or two or more antigen-binding fragments selected from the group consisting of: Fv, Fab, scFv and nanobody;
preferably, the C-terminus or N-terminus of the heavy chain of the anti-RAGE antibody is fused to the N-terminus or C-terminus of the scaffold protein or a truncation or variant thereof, alternatively, the anti-RAGE antibody is embedded in the sequence of the scaffold protein or a truncation or variant thereof, alternatively, the C-terminus or N-terminus of the antigen-binding fragment of the anti-RAGE antibody is fused to the N-terminus or C-terminus of the scaffold protein or a truncation or variant thereof, alternatively, the antigen-binding fragment of the anti-RAGE antibody is embedded in the sequence of the scaffold protein or a truncation or variant thereof.

17. A nucleic acid molecule, encoding the anti-RAGE antibody according to any one of items 1-6.

18. An expression vector, comprising the nucleic acid molecule according to item 17.

19. A cell, comprising the nucleic acid molecule according to item 17 or the expression vector according to item 18.

20. An extracellular vesicle, virus, liposome, cell, organelle or non-biological material, displaying the anti-RAGE antibody according to any one of items 1-6.

21. A method for displaying the anti-RAGE antibody according to any one of items 1-6 on extracellular vesicles, comprising:
performing fusion expression of a scaffold protein or a truncation or variant thereof with the anti-RAGE antibody according to any one of items 1-6 on the membrane of extracellular vesicles; or
connecting the anti-RAGE antibody according to any one of items 1-6 to the membrane of extracellular vesicle via an affinity pairing molecule; or
connecting the anti-RAGE antibody according to any one of items 1-6 to the membrane of extracellular vesicle by click chemistry reagents or by covalent bonding; or
embedding the anti-RAGE antibody according to any one of items 1-6 in the membrane of extracellular vesicle by lipophilic group.

22. A pharmaceutical composition, comprising the extracellular vesicle, virus, liposome, cell, organelle or non-biological material according to item 20, and a pharmaceutically acceptable carrier.

23. Use of the anti-RAGE antibody according to any one of items 1-6, the conjugate according to item 7 or 8, the bispecific or multispecific antibody according to item 9, the immune cell according to item 10 or 11, the antibody modified with lipophilic group according to item 12, the fusion protein according to any one of items 13-16, the extracellular vesicle, virus, liposome, cell, organelle or non-biological material according to item 20, or the pharmaceutical composition according to item 22, in the preparation of a medicament for diagnosing, treating and/or preventing a disease.

24. A method for diagnosing, treating or preventing a disease, comprising administering to a subject in need thereof an effective amount of the extracellular vesicle, virus, liposome, cell, organelle or non-biological material according to item 20 or the pharmaceutical composition according to item 22.

25. A method for diagnosing a disease, comprising contacting the extracellular vesicle, virus, liposome, cell, organelle or non-biological material according to item 20 or the pharmaceutical composition according to item 22 with a sample to be tested to detect RAGE antigen or a fragment thereof.

26. The method according to item 25, wherein the RAGE antigen or a fragment thereof is detected by flow cytometry.

### Effects of the Invention

(1) The anti-RAGE antibody according to the present application is a molecule with cross-reactivity among human, murine and monkey, which can target the RAGE antigen target of different species such as human, murine and monkey, and has high application value both in scientific research and clinical application.

(2) The present application realizes the display of anti-RAGE antibody on the surface of EVs for the first time; the anti-RAGE antibody according to the present application are applied to display on extracellular vesicle, which endows the extracellular vesicles with targeting ability, thereby ultimately enriching the extracellular vesicles in specific lesion tissues or organs (such as the lungs) with high expression of the RAGE antigen, and facilitating the delivery, release and therapeutic effects of other drug molecules loaded on the extracellular vesicles in the specific lesion tissues.

(3) The present application uses the extracellular vesicle modified by anti-RAGE antibody as a delivery carrier, which can achieve targeted delivery to tissues or organs with high expression of RAGE antigen. Due to the characteristics of the extracellular vesicles themselves, the delivery carriers have high safety, good biocompatibility, low immunogenicity, diversity of loaded drug molecules, and protection on drug molecules inside the vesicles (the phospholipid bilayer can protect the biological activity of the internal substances).

### BRIEF DESCRIPTION OF THE DRAWINGS

FIG. 1A shows the results of Coomassie brilliant blue staining on three His-tagged antigens;

FIG. 1B shows the results of Coomassie brilliant blue staining on three Fc-tagged antigens.

FIG. 2A shows the result of Coomassie brilliant blue staining on the h11E6.16 antibody; FIG. 2B shows the result of Coomassie brilliant blue staining on the control antibody.

FIG. 3 is an HPLC-SEC chromatogram of the h11E6.16 antibody.

FIG. 4A shows the binding activity of different antigens to h11E6.16 antibody (P38411); FIGS. 4B-4C show the binding activities of different antigens to anti-hRAGE (P46085).

FIG. 5 shows the detection results of the biotin labeling effect of RAGE antigen.

FIG. 6A shows the results of the activity analysis of the Fc-tagged RAGE antigen binding antibody before and after biotin labeling; FIG. 6B shows the results of the activity analysis of the His-tagged RAGE antigen binding antibody before and after biotin labeling.

FIG. 7A shows the flow cytometry result of a high-expressing RAGE cell line constructed by transiently transferring plasmid p318 into 293F cells; FIGS. 7B-7D are Western Blot detection results of a high-expressing RAGE cell line constructed by transiently transferring plasmid p318 into 293F cells; FIG. 7E is a confocal microscope photograph of a high-expressing RAGE cell line constructed by transiently transferring plasmid p318 into 293F cells.

FIG. 8A shows the flow cytometry result of a high-expression RAGE cell line constructed by transiently transferring plasmid p605 into HepG2 cells; FIGS. 8B-8D are Western Blot detection results of a high-expression RAGE cell line constructed by transiently transferring plasmid p605 into HepG2 cells.

FIGS. 9A-9B are flow cytometry results showing the specific binding ability of each candidate phage antibody pool to cells expressing human RAGE antigens.

FIGS. 10A-10L are Elisa results showing the specific binding ability of each candidate phage antibody pool to RAGE antigens of different species.

FIGS. 11A-11B are flow cytometry results showing the binding ability of each candidate antibody on p318-293F cells.

FIGS. 12A-12L show the analysis results of binding activities of the supernatant of eukaryotically expressed antibodies with the antigens of human-RAGE-ECD-Fc (P38406), cyno-RAGE-ECD-Fc (P38408), and mouse-RAGE-ECD-Fc (P38410).

FIG. 13A is flow cytometry result showing the binding ability of the antiRAGE-scFv antibody according to the present application to p318-293F cells and 293F cells; FIG. 13B are flow cytometry results of the specific binding of the antiRAGE-Fc antibody according to the present application to HepG2 cells transiently transfected with p605.

FIGS. 14A-14D respectively show the fluorescence intensity results of flag antibodies detected by nanoflow cytometry after using FITC (Proteintech/FITC-66008)-conjugated anti-flag antibodies to label p641 EV, p642 EV, and wild-type 293F EV, and using FITC (Proteintech/FITC-65128)-conjugated isotype control antibodies to label 293F EV.

FIGS. 15A-15D respectively show the fluorescence intensity results of HA antibodies detected by nanoflow cytometry after using CoraLite 488-conjugated anti-HA antibodies to label EVs displaying antiRAGE(A90)-scFv antibodies and antiRAGE(A90)-Fc antibodies on their surfaces, and using CoraLite 488-conjugated isotype control antibodies to label 293F EVs.

FIG. 16A shows the FITC mean fluorescence intensity (MFI) results of each group of EVs obtained by co-incubating A90-Fc EVs and control EVs labeled with FITC with p605 cells and HepG2 cells respectively, and FIG. 16B shows the verification results of the specific targeting ability of antiRAGE EVs to p605 cells.

### DETAILED DESCRIPTION

Particular embodiments of the present application will be described in more detail below with reference to the accompanying drawings. Although particular embodiments of the present application are shown in the drawings, it should be understood that the present application may be implemented in various forms and should not be limited to the embodiments set forth herein. On the contrary, these embodiments are provided to enable a more thorough understanding of the present application and to fully convey the scope of the present application to those skilled in the art.

It should be noted that, the words “comprise/include” or “comprising/including” mentioned throughout the specification and claims are open-ended terms, and should be interpreted as “including but not limited to”. The preferred embodiments are described subsequently in the specification for implementing the present application, and the description is for the purpose of illustrating the general principles of the present application and is not intended to limit the scope of the present application. The protection scope of this application shall be determined by the appended claims.

### Definitions

Unless defined otherwise, all technical and scientific terms used herein have the same meanings as understood by one of ordinary skill in the art.

As used herein, the term “receptor for advanced glycation end products (RAGE, or AGER)” refers to a multi-ligand cell surface member of the immunoglobulin superfamily. RAGE consists of an extracellular domain, a single transmembrane domain and a cytoplasmic tail. The extracellular domain of the receptor consists of one V-type immunoglobulin domain and two C-type immunoglobulin domains. RAGE is expressed by a variety types of cells, such as endothelial, smooth muscle cells, macrophages and lymphocytes in many different tissues, including lung, heart, kidney, skeletal muscle, and brain.

As used herein, the term “antibody” refers to a type of immunoglobulin that can specifically bind to an antigen. Such molecules typically comprise two heavy chains (H) and two light chains (L) interconnected by disulfide bonds. Each heavy chain is composed of a heavy chain variable region (VH) and a heavy chain constant region. The heavy chain constant region consists of three domains: CH1, CH2 and CH3. Each light chain is composed of a light chain variable region (VL) and a light chain constant region. The light chain constant region consists of one domain CL. The variable regions of the antibody heavy and light chains comprise the binding domain that interacts with the antigen. The constant regions of the antibodies may mediate the binding of an immunoglobulin to host tissues or factors, including various cells of the immune system (e.g., effector cells) and components of the complement system, such as C1q (the first component in the classical pathway of complement activation).

The heavy chain of an antibody may be divided into three functional regions: the Fd region, the hinge region and the Fc (fragment crystallizable) region. The Fd region comprises the VH and CH1 domains and combines with the light chain to form Fab (antigen-binding fragment). The Fc fragment is responsible for immunoglobulin effector functions including, for example, complement fixation and binding to homologous Fc receptors on effector cells. The hinge region found in IgG, IgA and IgD immunoglobulin classes acts as a flexible spacer, allowing the Fab portion to move freely in space relative to the Fc region. Hinge domains are structurally diverse, varying in sequence and length between immunoglobulin classes and between immunoglobulin subclasses.

The “light chain variable region” (VL) and the “heavy chain variable region” (VH) both comprise the following framework regions (FR) and CDR regions from amino-terminus to carboxyl-terminus: FR1, CDR1, FR2, CDR2, FR3, CDR3 and FR4. In this application, the CDR1, CDR2 and CDR3 of the light chain variable region are also referred to as CDR-L1, CDR-L2 and CDR-L3, respectively; and the CDR1, CDR2 and CDR3 of the heavy chain variable region are also referred to as CDR-H1, CDR-H2 and CDR-H3, respectively.

CDRs may be determined according to the Kabat definition, the Chothia definition, a cumulative of both the Kabat definition and the Chothia definition, the AbM definition, the contact definition, the IMGT unique numbering definition, and/or the conformational definition, or any CDR determination method well known in the art. In this application, the CDR sequences are determined by the Kabat definition.

Based on the amino acid sequence of the constant region of the antibody heavy chain, immunoglobulin molecules may be divided into five classes (isotypes): IgA, IgD, IgE, IgG and IgM, and may be further divided into different subtypes, such as IgG1, IgG2, IgG3, IgG4, IgA1 and IgA2, etc. Based on the amino acid sequence of the light chain, the light chains of antibodies may be divided into lambda (λ) chains and kappa (κ) chains.

As used herein, the term “antibody” should be understood in its broadest sense, including but not limited to: monoclonal antibodies, polyclonal antibodies, antigen-binding fragments, and the like. The antibodies may comprise additional modifications, such as the introduction of non-naturally occurring amino acids, mutations in the Fc region, and mutations in glycosylation sites. Antibodies also include post-translationally modified antibodies, fusion proteins comprising antigenic determinants of antibodies, and immunoglobulin molecules comprising any other modifications to the antigen recognition site, as long as these antibodies exhibit the desired biological activity.

As used herein, the term “antigen-binding fragment” may be produced by recombinant DNA techniques or by enzymatic or chemical cleavage of intact antibodies. Examples of antigen-binding fragments described herein include, but are not limited to: (1) an Fv fragment having the VL and VH domains of a single arm of an antibody; (2) a Fab fragment having VL, CL, VH and CH1 domains; (3) a Fab′ fragment, i.e., a Fab fragment having one or more cysteine residues at the C-terminus of the CH1 domain; (4) a F(ab′)2 fragment, i.e., a bivalent fragment comprising two Fab′ fragments linked by a disulfide bridge at the hinge region; (5) a Fd fragment having VH and CH1 domains; (6) a dsFv fragment, i.e., a disulfide-stabilized Fv antibody; (7) a scFv fragment consisting of a heavy chain variable region connected to a light chain variable region; (8) a sc(Fv)2 fragment, a single-chain antibody consisting of four variable regions, i.e., two light chain variable regions (VL) and two heavy chain variable regions (VH), connected by linkers; (9) a dAb fragment consisting of a VH domain; (10) isolated complementary determining regions; (12) antibodies with specific binding domains; (13) single domain antibodies as antibody fragments that comprise only a single variable domain of the entire antibody (such as nanobodies, which are single domain antibodies with only a heavy chain variable region); (14) domain-deleted antibodies; (15) CDR-grafted antibodies, which refer to antibodies that comprise heavy chain variable region and light chain variable region sequences from one species, but in which the sequences of one or more CDR regions of VH and/or VL are replaced with CDR sequences of another species, such as antibodies with murine heavy chain variable regions and light chain variable regions, in which one or more murine CDRs (e.g., CDR3) have been replaced with human CDR sequences; (16) diabodies, which are dimeric scFvs formed when the VH domain of a first scFv is assembled with the VL domain of a second scFv, and the VL domain of the first scFv is assembled with the VH domain of the second scFv, and the two antigen-binding domains of a diabody may be directed to the same or different epitopes; (17) Triabodies are trimeric scFvs formed in a manner similar to diabodies, but in which three antigen-binding domains are produced in a single complex; the three antigen-binding domains may be directed to the same or different epitopes; (18) tetrabodies are tetrameric scFvs formed in a manner similar to diabodies, but in which four antigen-binding domains are produced in a single complex; the four antigen-binding domains may be directed to the same or different epitopes; (19) minibodies are scFvs fused to a CH3 domain (see Olafsen et al., Protein Eng Des Sel., Vol. 17: 315-23, 2004).

As used herein, the term “bispecific antibody” refers to an artificial antibody that has fragments derived from two different monoclonal antibodies and is capable of binding to two different epitopes. The two epitopes may be present on the same antigen, or may be present on two different antigens.

As used herein, the term “multispecific antibody” refers to an antibody that specifically binds to at least two different antigens or at least two different epitopes of the same antigen. A multispecific antibody can bind to, for example, two, three, four, five or more different antigens, or can bind to two, three, four, five or more different epitopes of the same antigen.

As used herein, the term “monoclonal antibody (mAb)” or “mAb” or “Mab” refers to a homogeneous antibody pool, i.e., the individual antibodies that constitute the population are identical except for possible naturally occurring mutations and/or post-translational modifications (e.g., isomerizations, amidations) that may be present in minor amounts. Monoclonal antibodies are highly specific, being directed to a single antigen or epitope. The term “monoclonal” indicates the character of the antibody being obtained from a substantially homogeneous antibody pool, and is not to be interpreted as limiting the structure, origin, or preparation method of the antibody. In some embodiments, the monoclonal antibody is produced by a hybridoma method, a phage display method, a yeast display method, a recombinant DNA method, a single cell screening method, or a single cell sequencing method.

As used herein, the term “human antibody” is an antibody that has an amino acid sequence that corresponds to that of an antibody produced by a human, and/or has been prepared by any technique for preparing human antibodies. This definition of a human antibody specifically excludes a humanized antibody comprising non-human antigen-binding residues. Human antibodies may be produced by a variety of techniques known in the art, including: phage display libraries (Hoogenboom and Winter, 1991, J. Mol. Biol. 227:381; Marks et al., 1991, J. Mol. Biol. 222:581), and yeast display libraries (Chao et al., 2006, Nature Protocols 1:755-68). Methods for preparing human monoclonal antibodies are also described in: Cole et al., Monoclonal Antibodies and Cancer Therapy 77 (1985); Boerner et al., 1991, J. Immunol. 147 (1): 86-95; and van Dijk and van de Winkel, 2001, Curr. Opin. Pharmacol. 5:368-74. Human antibodies may be prepared by administering an antigen to a transgenic animal (e.g., a mouse) that has been modified to produce such antibodies in response to antigenic challenge but whose endogenous loci have been disabled (see, e.g., Jakobovits, 1995, Curr. Opin. Biotechnol. 6(5):561-66; Bruggemann and Taussing, 1997, Curr. Opin. Biotechnol. 8(4):455-58, and U.S. Pat. Nos. 6,075,181 and 6,150,584 related to XENOMOUSE™ technology). For human antibodies produced by human B cell hybridoma technology, see also, for example, Li et al., 2006, Proc. Natl. Acad. Sci. USA 103:3557-62.

As used herein, the term “chimeric antibody” refers to an antibody that combines antibody fragments from different species. Particularly, for example, the Fc constant region of a monoclonal antibody derived from one species (e.g., mouse) is replaced with an Fc constant region derived from another species (e.g., human) via DNA recombination technology. See, for example, patent applications PCT/US86/02269; and EP/173,494.

As used herein, the term “humanized antibody” refers to an antibody comprising a human immunoglobulin framework region and one or more CDRs from a non-human (e.g., mouse, rat, rabbit, or synthetic) immunoglobulin. Except for CDRs, all other parts in the humanized antibody are substantially identical to the corresponding parts of natural human immunoglobulin sequences. As for methods for constructing humanized antibodies by genetic engineering, see, for example, patent application US/5,585,089.

As used herein, the term “specific binding” refers to the property of complementary binding with high affinity determined by the antigenic determinant and the spatial conformation of the variable region of the antibody molecule. This high affinity determines that once the antibody molecule binds to the antigen, it can exert its corresponding physiological function. For example, in some embodiments of the present application, the antibody has the function of binding to and helping to clear the antigen.

As used herein, the term “human universal framework” refers to a framework that represents the amino acid residues that are most commonly found in the selected human immunoglobulin VL or VH framework sequences. Generally, the human immunoglobulin VL or VH sequence is selected from a subgroup of variable domain sequences. Generally, a subgroup of sequences is a subgroup as described in Kabat et al., Sequences of Proteins of Immunological Interest, 5th ed., NIH Publication 91-3242, Bethesda MD (1991), Vols. 1-3. In some embodiments, for the VL, the subgroup is a subgroup as described by Kabat et al. (supra). In some embodiments, for VH, the subgroup is subgroup III as described by Kabat et al. (supra).

As used herein, the term “homology” or “identity” refers to the percentage of amino acid residues or nucleotide residues that are identical between two sequences. If the two sequences to be compared to each other differ in length, sequence “homology” or “identity” preferably refers to the percentage of nucleotide residues in the shorter sequence that are identical to the amino acid residues or nucleotide residues in the longer sequence. Sequence identity may be routinely determined by sequence analysis software commonly used in the art, such as the Wisconsin Sequence Analysis Package and the like.

As used herein, the term “conjugate” (also referred to as “antibody-drug conjugate, ADC”) refers to a conjugate formed by covalently coupling a therapeutic agent to an antibody directly or indirectly via one or more appropriate linkers. ADCs are typically in the form of antibody-linker-therapeutic agent conjugates. An antibody-therapeutic agent conjugate combines the ideal properties of both an antibody and a therapeutic agent (a cytotoxic molecule or a substance with other property) to enhance their anti-tumor (or other pharmaceutical) activity by targeting tumor cells (or other cells/organs) expressing antigens with potent therapeutic agents (cytotoxic molecules or substances with other properties). The design intention of ADCs is to distinguish healthy cells from diseased tissue, such as tumor cells in a tumor.

As used herein, the term “therapeutic agent” refers to any cytotoxic molecule having, for example, an anti-tumor effect, an anti-infective or anti-inflammatory effect and having at least one substituent group or moiety structure that allows attachment to a linker structure. Therapeutic agents can kill cells (e.g., cancer cells) and/or inhibit the growth, proliferation, or metastasis of cells (e.g., cancer cells), thereby reducing, ameliorating, or eliminating one or more symptoms of a disease or disorder (e.g., cancer).

As used herein, the term “vector” generally refers to a nucleic acid molecule capable of self-replication in a suitable host, which transfers an inserted nucleic acid molecule into and/or between host cells. The vector may include a vector primarily used to insert DNA or RNA into cells, a vector primarily used to replicate DNA or RNA, and a vector primarily used for expression by transcription and/or translation of DNA or RNA. The vector also includes a vector having multiple functions as described above. The vector may be a polynucleotide that may be transcribed and translated into a polypeptide when introduced into an appropriate host cell. Generally, the vector can produce the desired expression product by culturing appropriate host cells comprising the vector.

As used herein, the terms “nucleic acid” or “polynucleotide” or “nucleic acid molecule” generally refer to deoxyribonucleic acid (DNA) or ribonucleic acid (RNA) and polymers thereof in either single- or double-stranded form. Unless specifically limited, the term can include nucleic acids comprising analogs of natural nucleotides that have similar binding properties as a reference nucleic acid (e.g., showing sequence information) and are metabolized in a manner similar to naturally occurring nucleotides. Unless otherwise indicated, the sequence of a nucleic acid may include conservatively modified variants thereof, such as substitutions of degenerate codon, alleles, orthologues, SNPs, and complementary sequences, as well as an explicitly indicated sequence.

As used herein, the term “plasmid” refers to a DNA molecule other than chromosomes (or nucleoids) in organisms such as bacteria, yeast and actinomycetes. It exists in the cytoplasm or nucleus and has the ability to replicate autonomously, so that it can maintain a constant copy number in daughter cells and express the genetic information it carries.

As used herein, the term “extracellular vesicle,” “exovesicle,” or “EV” refers to a cell-derived vesicle comprising a membrane enclosing an interior space. Extracellular vesicles include all membrane-bound vesicles (e.g., exosomes, microvesicles, apoptotic bodies, oncosomes, nanovesicles, etc.) whose diameters are smaller than those of the cells from which they originate. In some aspects, the diameter of the extracellular vesicle ranges from 20 nm to 1000 nm, and can comprise various macromolecular payloads within the interior space (i.e., lumen), displayed on the outer surface of the extracellular vesicle, and/or across the membrane. In some aspects, the payload can include nucleic acids, proteins, carbohydrates, lipids, small molecules, and/or combinations thereof. In certain aspects, the extracellular vehicle comprises a scaffold portion. By way of example and not limitation, extracellular vesicles include apoptotic bodies, cell debris, cell-derived vesicles obtained by direct or indirect manipulation (e.g., by serial extrusion or treatment with alkaline solutions), vesicular organelles, and vesicles produced by living cells (e.g., by direct budding from the plasma membrane or fusion of late endosomes with the plasma membrane). Extracellular vesicles may be derived from living or dead organisms, explanted tissues or organs, prokaryotic or eukaryotic cells, and/or cultured cells. In some aspects, extracellular vesicles are produced by cells expressing one or more transgene products.

As used herein, the term “affinity pairing molecule” refers to a molecule obtained by combining molecule A and molecule B with a strong affinity effect.

As used herein, the term “transmembrane protein” refers to a protein that spans across a biological membrane. Many transmembrane proteins function as channels or “loading docks” to deny or allow the certain substances to transport across the biological membrane and enter the cell, and also allow the waste byproducts to be transported out of the cell. When responding to a certain molecule, these “carrying” transmembrane proteins transport the molecule across the biological membrane through specific folding and bending methods.

As used herein, the term “spy tag/spy catcher” refers to a biochemical reaction pair derived from a protein tool pair of Streptococcus pyogenes. The tag part is a short peptide of only a dozen amino acids, while the catcher part is a longer protein domain. They have good biocompatibility, and can react efficiently to form isopeptide bonds under physiological conditions.

As used herein, the term “click chemistry”, also known as “linking chemistry” and “speed-matching combinatorial chemistry”, is a synthetic concept introduced by chemist Karl Barry Sharpless in 2001. The main purpose is to quickly and reliably complete the chemical synthesis of various molecules by splicing small units. It places particular emphasis on developing new combinatorial chemistry methods based on carbon-heteroatom bond (C-X-C) synthesis and using click reactions to simply and efficiently obtain molecular diversity. The representative reaction of click chemistry is the copper-catalyzed azide-alkyne Husigen cycloaddition reaction. The reagents participating in click chemistry reactions are called click chemistry reagents.

As used herein, the term “treatment” refers to any intervention that results in any observable beneficial effect of treatment, or any statistically significant indicator of success in treating or ameliorating a disease or condition, such as improving the signs, symptoms, or progression of a disease or pathological condition. For example, a beneficial effect may be demonstrated by a decrease in disease, a delay in the onset of disease or a lessening in severity of clinical symptoms of disease in a subject, a decrease in the frequency with which a subject experiences disease symptoms, a slowing of the progression of disease, a decrease in the number of disease relapses, an improvement in the subject's overall health, or by other parameters specific to a particular disease.

As used herein, the term “prevention” is treatment administrated to a subject who does not show signs of a disease or who shows only early signs of the disease, with the goal of reducing the risk of developing the pathology or further progression of the early disease.

The term “administrate/administrating” as used herein refers to parenteral, intravenous, intraperitoneal, intramuscular, intratumoral, intralesional, intranasal or subcutaneous administration, oral administration, administration by suppository, topical contact, intrathecal administration or implantation of a sustained release device, such as a mini-osmotic pump, to a subject in need thereof.

As used herein, the term “a subject in need thereof” refers to an individual who is at risk of or suffering from a disease, disorder or condition, and who may be treated or improved by the pharmaceutical composition or extracellular vesicle described herein. As used herein, the term “subject in need thereof” refers to a mammal, such as a human being, but it may also be other animal, such as wild animal (e.g., heron, stork, crane, etc.), domestic animal (e.g., duck, goose, etc.), or experimental animal (e.g., gorilla, monkey, rat, mouse, rabbit, guinea pig, marmot, ground squirrel, etc.).

The terms “effective amount” or “effective dose” as used herein refer to an amount of a pharmaceutical composition or an extracellular vesicle sufficient to achieve a desired (e.g., beneficial) effect in a subject treated with the pharmaceutical composition or the extracellular vesicle, such as an amount sufficient to statistically significantly improve one or more symptoms of the disease being treated, to statistically significantly slow the deterioration of a progressive disease, or to statistically significantly prevent the onset of other related symptoms or diseases, or any combination thereof. In some embodiments, an effective amount of a pharmaceutical composition or extracellular vesicle is an amount sufficient to inhibit or treat a disease with minimal or no toxicity in a subject, excluding the presence of one or more adverse side effects. An effective amount or dose may be administrated once or multiple times over a given period of time. The effective amount or dosage may depend on the purpose of the treatment, and may be determined by one skilled in the art according to the needs of the subject. When referring to an individual active ingredient administrated alone, the effective amount or dosage refers to that ingredient alone. When referring to a combination, an effective amount or dosage refers to combined amounts of the active ingredients that produce a therapeutic effect, whether administrated serially or simultaneously.

### Anti-RAGE Antibody

The present application provides an anti-RAGE antibody, which comprises three heavy chain complementary determining regions (CDR-H) and three light chain complementary determining regions (CDR-L), wherein the three heavy chain complementary determining regions are CDR-H1, CDR-H2 and CDR-H3, and the three light chain complementary determining regions are CDR-L1, CDR-L2 and CDR-L3, wherein
the amino acid sequence of the CDR-H1 is represented by SEQ ID NO: 1 (NNYAIN);
the amino acid sequence of the CDR-H2 is represented by SEQ ID NO: 2 (RIVPFFDVTN);
the amino acid sequence of the CDR-H3 is represented by SEQ ID NO: 3 (GSFNWNSGAFDI);
the amino acid sequence of the CDR-L1 is represented by SEQ ID NO: 4 (QSISSWLA);
the amino acid sequence of the CDR-L2 is represented by SEQ ID NO: 5 (IYQASSLES); and
the amino acid sequence of the CDR-L3 is represented by SEQ ID NO: 6 (QYKDYPLT).

In some embodiments, the anti-RAGE antibody comprises a human universal framework region. In some embodiments, the VH comprises a universal framework region (FR) of human subgroup III. In some embodiments, the VL comprises a universal framework of human subgroup. In some embodiments, it comprises a human universal framework region (FR) and one or more amino acid substitutions based on the human universal framework region (FR).

In some embodiments, the framework region is a human universal framework region, and comprises one or more (e.g., 1-20, 1-15, 1-10, 1-5, 1-4, 1-3) amino acid substitutions, deletions, or insertions. In some embodiments, the framework region is a human universal framework region, or have 70%, 80%, 90%, 95%, 97%, 98%, 99% identity with the human universal framework region.

In some embodiments, the antibody comprises a heavy chain variable region and a light chain variable region, wherein
the amino acid sequence of the heavy chain variable region is represented by SEQ ID NO:7 (QVQLVQSGAEVKKPGSSVKVSCKASGGTFNNYAINWARQAPGQGLEWMGRIVPFFDV TNYAQKFQGRVTITADKSTSTAYMELSSLRSEDTAIYYCAIGSFNWNSGAFDIWGQGTP VTVSS), or an amino acid sequence having at least 90%, 91%, 92%, 93%, 94%, 95%, 96%, 97%, 98% or 99% identity with SEQ ID NO:7; and
the amino acid sequence of the light chain variable region is represented by SEQ ID NO: 8 (NIQLTQSPSTLSASVGDRVTITCRASQSISSWLAWYQQKPGKAPKLLIYQASSLESGVPS RFSGSGSGTEFTLTIRSLQPEDFAVYFCQQYKDYPLTFGGGTKVDIK), or an amino acid sequence having at least 90%, 91%, 92%, 93%, 94%, 95%, 96%, 97%, 98%, or 99% identity with SEQ ID NO: 8.

In some embodiments, the anti-RAGE antibody is a human antibody, a humanized antibody, or a chimeric antibody.

In some embodiments, the anti-RAGE antibody is a full-length IgG antibody, preferably a full-length IgG1 antibody.

In some embodiments, the anti-RAGE antibody is any one antigen binding fragment selected from the group consisting of: Fv, Fab, F(ab′)2, Fab′, Fd, dsFv, scFv, sc(Fv)2, dAb, VH-CH1-CH2-CH3, VH-CH1, scFv-Fc, isolated complementary determining region, domain-specific antibody, single domain antibody, domain-deleted antibody, CDR-grafted antibody, diabody, triabody, tetrabody and minibody.

In some embodiments, the anti-RAGE antibody comprises a heavy chain and a light chain, wherein the heavy chain configuration is VH-CH1 and the light chain configuration is VL-CL (Kappa).

In the above embodiments, the VH sequence is represented by SEQ ID NO: 7, and the CH1 sequence is represented by SEQ ID NO: 9 (ASTKGPSVFPLAPSSKSTSGGTAALGCLVKDYFPEPVTVSWNSGALTSGVHTFVLQSSG LYSLSSVVTVPSSSLGTQTYICNVNHKPSNTKVDKKVEPKSCDKTHTCPPCPELLGGPSV FLFPPKPKDTLMISRTPEVTCVVVDVSHEDPEVKFNWYVDGVEVHNAKTKPREEQYNST YRVVSVLTVLHQDWLNGKEYKCKVSNKALPIEKTISKAKGQPREPQVYTLPPSRDELTK NQVSLTCLVKGFYPSDIAVEWESNGQPENNYKTTPPVLDSDGSFFLYSKLTVDKSRWQQ GNVFSCSVMHEALHNHYTQKSLSLSPGK); the CL (Kappa) sequence is represented by SEQ ID NO: 10 (RTVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC), and the VL sequence is represented by SEQ ID NO: 8.

In some embodiments, the anti-RAGE antibody binds to RAGE antigen of human, monkey or mouse. The anti-RAGE antibody is a molecule with cross-reactivity among human, murine and monkey, and has wider applicability both in scientific research and clinical practice. In some embodiments, the murine is a rat or a mouse. In some embodiments, the monkey includes but is not limited to an animal of the genus Macaca.

### Conjugate

The present application further provides a conjugate comprising any one of the above anti-RAGE antibodies.

In some embodiments, the conjugate further comprises a therapeutic agent conjugated to the anti-RAGE antibody. In some embodiments, the therapeutic agent is any one or more selected from the group consisting of: a cytotoxic agent, a hormone preparation, a small molecule targeting preparation, a proteasome inhibitor, an immunomodulator, an angiogenesis inhibitor, a cell proliferation inhibitor, a pro-apoptotic agent, a cytokine, and an activator of a co-stimulatory molecule.

Examples of cytotoxic agents include, but are not limited to: anthracycline, auristatin, CC-1065, dolastatin, duocarmycin, enediyne, geldanamycin, maytansine, puromycin, taxane, vinca alkaloid, SN-38, tubulysin, hemiasterlin, eribulin, trabectedin, lubinectedin, and a stereoisomer, analog, or derivative thereof.

Examples of hormone preparation include, but are not limited to: tamoxifen, onapristone, raloxifene (Evista), 4-hydroxytamoxifen, trioxifene, keoxifene, LY117018, flutamide and nilutamide; and a pharmaceutically acceptable salt, acid or derivative thereof.

Examples of cytokine include, but are not limited to: growth hormone, such as human growth hormone, N-methionyl human growth hormone and bovine growth hormone; parathyroid hormone; thyroxine; insulin; proinsulin; relaxin; prorelaxin; glycoprotein hormones (such as follicle stimulating hormone (FSH); thyroid stimulating hormone (TSH) and luteinizing hormone (LH)); hepatocyte growth factor; fibroblast growth factor; prolactin; placental lactogen; tumor necrosis factor α and β; mullerian-inhibiting substance; mouse gonadotropin-related peptide; inhibin; activin; vascular endothelial growth factor; integrin; thrombopoietin (TPO); nerve growth factor (such as NGF-β); platelet growth factor; transforming growth factors (TGFs) (such as TGF-α and TGF-β); insulin-like growth factor I and II; erythropoietin (EPO); osteoinductive factor; interferons (such as interferon α, β and γ); colony stimulating factors (CSFs) (such as macrophage-CSF (M-CSF); granulocyte-macrophage-CSF (GM-CSF) and granulocyte-CSF (G-CSF)); interleukins (ILs) (such as IL-1; IL-1α; IL-2; IL-3; IL-4; IL-5; IL-6; IL-7; IL-8; IL-9; IL-11; IL-12); tumor necrosis factor such as TNF-α or TNF-β; and other polypeptide factors (including LIF and kit ligand (KL)). As used herein, the term “cytokine” includes proteins from natural sources or from recombinant cell culture, as well as biologically active equivalents of the native sequence cytokines.

Examples of small molecule targeting preparation include, but are not limited to: erlotinib, gefitinib, osimertinib, afatinib, icotinib, dacomitinib, sorafenib, imatinib, and sunitinib.

Examples of proteasome inhibitor include, but are not limited to: bortezomib, ixazomib, carfilzomib, oprozomib, delanzomib, marizomib, and MG132.

Examples of immunomodulator include, but are not limited to: thymosin, levamisole, Toll-like receptor modulator, STING modulator.

Examples of angiogenesis inhibitor include, but are not limited to: bevacizumab, apatinib, anlotinib, sorafenib, and cetuximab.

A cell proliferation inhibitor refers to an agent that has a cytostatic effect on cells, thereby inhibiting the growth and/or expansion of a specific subset of cells.

Bispecific or Multispecific Antibody, Antibody Modified with Lipophilic Group, and Immune Cell

The present application further provides a bispecific or multispecific antibody comprising any one of the above anti-RAGE antibodies.

The bispecific antibody according to the present application comprises an anti-RAGE antibody or an antigen-binding fragment thereof, and another antibody or a fragment thereof, or an antibody analog.

In some embodiments, the multispecific antibody is a trispecific antibody or a tetraspecific antibody.

The multispecific antibody according to the present application comprises an anti-RAGE antibody or an antigen-binding fragment thereof, and two or three additional antibodies or fragments thereof, or antibody analogs.

The antibody belonging to the bispecific antibody or the multispecific antibody may be a scFv-based antibody, a Fab-based antibody, or an IgG-based antibody. A bispecific or multispecific antibody may inhibit or amplify two or more signals simultaneously, and are therefore more effective than a single antibody that inhibits/amplifies one signal. It may be administered at a lower dose compared with the case where each signal is treated with each signal inhibitor, and can inhibit/amplify two or more signals at the same time and space.

Methods for producing bispecific or multispecific antibodies are well known. Traditionally, the recombinant production of a bispecific antibody is based on the co-expression of two or more immunoglobulin heavy chain/light chain pairs, under conditions where the two or more heavy chains have different specificities.

The present application further provides an antibody modified with a lipophilic group, wherein the antibody is any one of the above anti-RAGE antibodies.

In some embodiments, the lipophilic group is any one or two or more selected from the group consisting of: 1,2-distearoyl-sn-glycero-3-phosphoethanolamine (DSPE), dioleoylphosphatidylethanolamine (DOPE), and cholesterol.

The present application further provides an immune cell comprising any one of the above anti-RAGE antibodies or nucleic acids encoding the antibody.

In some embodiments, the immune cell may be any one or more selected from the group consisting of: T cell, NK cell, cytokine-induced killer cell (CIK), activated cytotoxic T lymphocyte (CTL), macrophage, tumor infiltrating lymphocyte (TIL), and dendritic cell.

### Fusion Protein

The present application further provides a fusion protein comprising any of the above anti-RAGE antibodies and one or more polypeptides or proteins.

In some embodiments, the polypeptide or protein is a scaffold protein, or a truncation or variant thereof, an affinity pairing molecule, an antibody, or a cytokine.

In some embodiments, the affinity pairing molecule is any one selected from the group consisting of. NbALFA/ALFA (see Hansjorg Gotzke et al., “The ALFA-tag is a highly versatile tool for nanobody-based bioscience applications.” Nature Communications.), monomer streptavidin/biotin, Strep-tag II/Strep-Tactin, N-terminal intein (Intein N)/C-terminal intein (Intein C) (see Adam J. Stevens et al., “Design of a Split Intein with Exceptional Protein Splicing Activity. J. Am. Chem. Soc. (2016)”), Spy Tag/Spy Catcher (see Zakeri, Bijan et al., “Peptide tag forming a rapid covalent bond to a protein, through engineering a bacterial adhesin.” Proceedings of the National Academy of Sciences of the United States of America, 109.12(2012)), and Protein A/Fc domain, preferably NbALFA/ALFA.

As used herein, the term “truncation of a scaffold protein” refers to any fragment of the original sequence of scaffold protein.

As used herein, the term “variant of a scaffold protein” refers to a protein that differs from the original scaffold protein by addition, deletion or substitution of at least one amino acid residue. In some embodiments, a variant of a scaffold protein is distinguished from the original scaffold protein by one or more substitutions, which may be conservative or non-conservative. In some embodiments, a variant of a scaffold protein comprises conservative substitutions. Variants of scaffold proteins also include scaffold proteins in which one or more amino acids are added or deleted, or substituted with another amino acid residue.

In some embodiments, the scaffold protein is a transmembrane protein, and preferably the scaffold protein is any one or two or more selected from the group consisting of: PLXNA1, MFGE8, PTGFRN, BASP1 and PDGFR.

PLXNA1 is a type I transmembrane protein composed of several extracellular domains, transmembrane domains and intracellular domains. In some embodiments, the truncation of PLXNA1 is PLXNA1 (positions 863-1316 of the original sequence), PLXNA1 (positions 863-1300 of the original sequence), PLXNA1 (positions 960-1300 of the original sequence), PLXNA1 (positions 1046-1300 of the original sequence), or PLXNA1 (positions 1143-1300 of the original sequence), or comprises a sequence having at least 80%, 85%, 90%, 91%, 92%, 93%, 94%, 95%, 96%, 97%, 98%, 99% homology to any of the above five truncation sequences. In some embodiments, the MFGE8 truncate is MFGE8 (positions 70-387 of the original sequence), or comprises a sequence having at least 80%, 85%, 90%, 91%, 92%, 93%, 94%, 95%, 96%, 97%, 98%, 99% homology to MFGE8. In some embodiments, PTGFRN and BASP1 scaffold proteins or truncations thereof may be found in, for example, Dooley K et al., “A Versatile Platform for Generating Engineered Extracellular Vesicles with Defined Therapeutic Properties”, [J]. Molecular Therapy, 2021, 29(5). DOI:10.1016/j.ymthe.2021.01.020. For PDGFR, see, for example, Andreia M. Silva et al., “Quantification of protein cargo loading into engineered extracellular vesicles at single-vesicle and single-molecule resolution”, [J]. Journal of Extracellular Vesicles, 2021, 10(10). DOI:10.1002/jev2.12130.

In some embodiments, the antibody is any one or two or more selected from the group consisting of: Fv, Fab, F(ab′)2, Fab′, Fd, dsFv, scFv, sc(Fv)2, dAb, isolated complementary determining region, domain-specific antibody, single domain antibody, domain-deleted antibody, CDR-grafted antibody, diabody, triabody, tetrabody and minibody.

In some embodiments, the C-terminus or N-terminus of the heavy chain of the anti-RAGE antibody is fused to the N-terminus or C-terminus of the scaffold protein or a truncation or variant thereof. In some embodiments, the anti-RAGE antibody is embedded in the sequence of the scaffold protein or a truncation or variant thereof. In some embodiments, the C-terminus or N-terminus of the antigen-binding fragment of the anti-RAGE antibody is fused to the N-terminus or C-terminus of the scaffold protein or a truncation or variant thereof. In some embodiments, the antigen-binding fragment of the anti-RAGE antibody is embedded in the sequence of the scaffold protein or a truncation or variant thereof.

### Nucleic Acid Molecule, Expression Vector, Cell, and Extracellular Vesicle, Virus, Liposome, Cell, Organelle or Non-Biological Material Displaying Anti-RAGE Antibody

The present application further provides a nucleic acid molecule encoding any one of the above anti-RAGE antibodies.

In some embodiments, the sequence of the nucleic acid molecule encoding the heavy chain of the anti-RAGE antibody is represented by SEQ ID NO: 11, or a nucleotide sequence having at least 70%, 75%, 80%, 85%, 90%, 91%, 92%, 93%, 94%, 95%, 96%, 97%, 98% or 99% similarity to SEQ ID NO: 11; the sequence of the nucleic acid molecule encoding the light chain of the anti-RAGE antibody is represented by SEQ ID NO: 12, or a nucleotide sequence having at least 70%, 75%, 80%, 85%, 90%, 91%, 92%, 93%, 94%, 95%, 96%, 97%, 98% or 99% similarity to SEQ ID NO: 12.

Particularly, SEQ ID NO: 11 is as follows:

ATGGGTTGGAGCCTCATCTTGCTCTTCCTTGTCGCTGTTGCTACGCGTG
TCCACTCCCAGGTTCAGCTGGTGCAGTCTGGGGCTGAGGTGAAGAAGCC
TGGGTCCTCGGTGAAGGTCTCCTGCAAGGCTTCTGGAGGCACCTTCAAT
AACTATGCTATCAACTGGGCGCGACAGGCCCCTGGACAAGGGCTTGAGT
GGATGGGAAGGATCGTCCCTTTCTTTGATGTGACAAACTACGCACAGAA
ATTCCAGGGCAGGGTCACGATTACCGCGGACAAATCCACGAGCACAGCC
TACATGGAATTGAGCAGCCTGAGATCTGAGGACACGGCCATATATTACT
GTGCGATAGGGTCGTTTAACTGGAACTCGGGTGCTTTTGATATCTGGGG
CCAGGGGACCCCGGTCACCGTCTCATCAGCTTCCACCAAGGGCCCCTCC
GTGTTCCCCCTGGCTCCCTCTTCCAAGAGCACCAGCGGCGGCACCGCTG
CTCTGGGATGTCTGGTGAAGGACTACTTCCCTGAGCCTGTGACCGTGTC
CTGGAATTCCGGCGCCCTGACCTCCGGCGTGCACACATTCCCTGCTGTG
CTGCAGTCCTCCGGCCTGTATAGCCTGTCCTCCGTGGTGACAGTGCCTA
GCTCCAGCCTGGGCACCCAGACCTATATCTGCAACGTGAACCACAAGCC
TAGCAATACCAAGGTGGACAAGAAGGTGGAGCCTAAGAGCTGCGACAAG
ACCCACACCTGTCCTCCATGTCCTGCTCCAGAACTGCTCGGCGGACCTT
CCGTGTTCCTGTTTCCTCCAAAGCCTAAGGACACCCTGATGATCAGCAG
AACCCCTGAAGTGACCTGCGTGGTGGTGGATGTGTCCCACGAGGATCCC
GAAGTGAAGTTCAATTGGTACGTGGACGGCGTGGAAGTGCACAACGCCA
AGACCAAGCCTAGAGAGGAACAGTACAACAGCACCTACAGAGTGGTGTC
CGTGCTGACCGTGCTGCACCAGGATTGGCTGAACGGCAAAGAGTACAAG
TGCAAGGTGTCCAACAAGGCCCTGCCTGCTCCTATCGAGAAAACCATCA
GCAAGGCCAAGGGCCAGCCTAGGGAACCCCAGGTTTACACACTGCCTCC
AAGCAGGGACGAGCTGACCAAGAATCAGGTGTCCCTGACCTGCCTGGTC
AAGGGCTTCTACCCTTCCGATATCGCCGTGGAATGGGAGAGCAATGGCC
AGCCTGAGAACAACTACAAGACAACCCCTCCTGTGCTGGACAGCGACGG
CTCATTCTTCCTGTACAGCAAGCTGACAGTGGACAAGAGCAGATGGCAG
CAGGGCAACGTGTTCAGCTGCAGCGTGATGCACGAGGCCCTGCACAACC
ACTACACCCAGAAGTCCCTGAGCCTGTCTCCTGGCAAAGGAGGCGGAGG
CTCTTACCCTTACGATGTGCCTGATTACGCTGGCGGAGGCGGCAGCCCT
TCTAGACTGGAAGAAGAACTGCGGCGGAGACTGACCGAA

SEQ ID NO: 12 is as follows:

ATGGGTTGGAGCCTCATCTTGCTCTTCCTTGTCGCTGTTGCTACGCGTG
TCCACTCCAACATCCAGTTGACCCAGTCTCCTTCCACCCTGTCTGCATC
TGTAGGCGACAGAGTCACCATCACTTGCCGGGCCAGTCAGAGTATTAGT
AGCTGGTTGGCCTGGTATCAGCAGAAACCAGGGAAAGCCCCTAAACTCC
TGATCTATCAGGCGTCTAGTTTAGAAAGTGGGGTCCCATCAAGGTTCAG
CGGCAGTGGATCTGGGACAGAATTCACTCTCACCATCCGCAGCCTACAG
CCTGAAGATTTTGCAGTTTATTTCTGCCAACAGTATAAAGATTACCCTC
TCACTTTCGGCGGAGGGACCAAGGTGGATATCAAAAGGACCGTGGCTGC
CCCCAGCGTGTTCATCTTCCCTCCTAGCGACGAGCAGCTGAAGAGCGGC
ACCGCTAGCGTGGTGTGTCTGCTGAATAACTTCTATCCCAGGGAGGCCA
AGGTGCAGTGGAAGGTGGATAACGCCCTGCAGAGCGGCAACTCCCAGGA
GTCCGTGACCGAGCAGGACTCCAAGGACAGCACCTACTCCCTGAGCTCC
ACCCTGACCCTGTCCAAGGCTGATTATGAGAAGCACAAGGTGTATGCTT
GCGAGGTGACACACCAGGGCCTGTCCAGCCCTGTGACCAAGAGCTTCAA
CCGGGGCGAGTGC

The present application further provides an expression vector comprising any of the above nucleic acid molecules.

In some embodiments, the expression vector may be any type of plasmid, which refers to a circular double-stranded DNA loop into which additional DNA segments may be inserted, for example, by standard molecular cloning techniques.

The present application further provides a cell comprising any one of the above nucleic acid molecules or expression vectors.

The antibody of the present application may be displayed on any delivery carriers. Therefore, the present application further provides a delivery carrier displaying any of the above anti-RAGE antibodies, wherein the delivery carrier is an extracellular vesicle, virus, liposome, cell, organelle or non-biological source material.

In some embodiments, other drug molecules are loaded in the delivery carrier of the present application.

### Method for Displaying Anti-RAGE Antibodies on Extracellular Vesicles

The present application provides a method for displaying any one of the above anti-RAGE antibodies on extracellular vesicles, comprising: performing fusion expression of a scaffold protein or a truncation or variant thereof with any one of the above anti-RAGE antibodies on the extracellular vesicle membrane.

In some embodiments, the scaffold protein or a truncation or variant thereof is as described above.

In some embodiments, the method comprises: achieving fusion expression of the anti-RAGE antibody and molecule B by plasmid transfection.

In some embodiments, the PLXNA1 truncation is co-expressed with any one of the above anti-RAGE antibodies on the extracellular vesicle membrane.

The present application provides a method for displaying any one of the above anti-RAGE antibodies on extracellular vesicles, comprising: connecting any one of the above anti-RAGE antibodies to the extracellular vesicle membrane via an affinity pairing molecule.

In some embodiments, the affinity pairing molecule is as described above.

In some embodiments, the method comprises: recombinantly expressing a fusion protein of an anti-RAGE antibody and molecule B; constructing extracellular vesicles incorporating a scaffold protein fused to molecule A or a truncation or variant thereof by plasmid transfection; co-incubating the recombinant protein comprising molecule B with the extracellular vesicles fused with molecule A, wherein the affinity of molecule A and molecule B causes the anti-RAGE antibody to be displayed on the extracellular vesicle membrane.

The present application provides a method for displaying any one of the above anti-RAGE antibodies on extracellular vesicles, comprising: connecting any one of the above anti-RAGE antibodies to the extracellular vesicle membrane by click chemistry reagents or by covalent binding.

In some embodiments, the click chemistry reagents are as described above.

In some embodiments, any one of the above anti-RAGE antibodies can be linked to the extracellular vesicle membrane by click chemistry reagents with reference to the method described in “Surface Functionalization of Exosomes Using Click Chemistry”, [J]. Bioconjugate Chemistry, 2014, 25(10):1777-1784. DOI:10.1021/bc500291r.

In some embodiments, any one of the above anti-RAGE antibodies can be linked to the extracellular vesicle membrane by covalent binding with reference to the method described in “Covalent conjugation of extracellular vesicles with peptides and nanobodies for targeted therapeutic delivery”, [J]. Journal of Extracellular Vesicles, 2021, 10(4). DOI:10.1002/jev2.12057.

The present application provides a method for displaying any one of the above anti-RAGE antibodies on extracellular vesicles, comprising: embedding any one of the above anti-RAGE antibodies in the extracellular vesicle membrane by a lipophilic group.

In some embodiments, the lipophilic group is as described above.

In some embodiments, any one of the above anti-RAGE antibodies can be embedded in the extracellular vesicle membrane by a lipophilic group with reference to the method described in “Display of GPI-anchored anti-EGFR nanobodies on extracellular vesicles promotes tumour cell targeting”, Sander et al. Journal of Extracellular Vesicles 5.1(2016):31053.”.

Pharmaceutical Composition, Pharmaceutical Use, and Method for Diagnosing, Treating and/or Preventing Diseases

The present application provides a pharmaceutical composition comprising an extracellular vesicle, virus, liposome, cell, organelle or non-biological material displaying any one of the above anti-RAGE antibodies, and a pharmaceutically acceptable carrier.

In some embodiments, the pharmaceutically acceptable carrier may be selected from the group consisting of: water, buffered aqueous solution, isotonic saline solution such as PBS (phosphate buffered saline), glucose, mannitol, dextrose, lactose, starch, magnesium stearate, cellulose, magnesium carbonate, 0.3% glycerol, hyaluronic acid, ethanol or polyalkylene glycol such as polypropylene glycol, triglycerides, etc.

In some embodiments, the pharmaceutical composition according to the present application may further comprise a lubricant, preservative, stabilizer, wetting agent, emulsifier, salt that affects osmotic pressure, buffer, coloring substance, etc. as additives. The pharmaceutical composition according to the present application may be administered orally or parenterally.

Parenteral administration may be intravenous injection, subcutaneous injection, intramuscular injection, intraperitoneal injection, endothelial administration, topical administration, intranasal administration, pulmonary administration, rectal administration, etc.

When administered orally, since proteins or peptides are digested in the stomach, oral compositions should be formulated by protecting the active drug with enteric coating to prevent its degradation in the stomach. Furthermore, the pharmaceutical composition may be administered by using any device capable of delivering the active substance to the target cells.

The appropriate dosage of the pharmaceutical composition according to the present application may vary based on factors such as formulation method, administration method, and the patient's age, weight, sex, pathological condition, diet, administration time, administration route, excretion rate and responsiveness, and an ordinary physician with ordinary skills can easily determine and prescribe an effective dosage for the desired treatment or prevention.

The pharmaceutical composition according to the present application may be prepared into a unit dosage form, or may be incorporated into a multi-dose container by formulating together with a pharmaceutically acceptable carrier and/or excipient according to a method that may be easily performed by a person skilled in the art. Herein, the preparation may be in the form of a solution, suspension or emulsion in an oil or aqueous medium, or may be in the form of an extract, powder, suppository, granule, tablet or capsule. The composition may further comprise a dispersant or a stabilizer.

The present application further provides use of any one of the above anti-RAGE antibodies, conjugates, bispecific or multispecific antibodies, immune cells, antibodies modified with lipophilic group, fusion proteins, extracellular vesicles, viruses, liposomes, cells, organelles or non-biological materials or pharmaceutical compositions in the preparation of a medicament for diagnosing, treating and/or preventing diseases.

The present application further provides a method for diagnosing, treating and/or preventing a disease, comprising: administering an effective amount of any one of the above extracellular vesicles, viruses, liposomes, cells, organelles or non-biological materials or pharmaceutical compositions to a subject in need thereof.

For example, the disease is an immune disease or cancer, and the cancer may be lung cancer.

### EXAMPLES

### Example 1: Construction of RAGE Antigen Proteins and Antigen Cells

1.1 RAGE Antigen Proteins from Human, Monkey and Murine were Synthesized for Antibody Screening.

### Experimental Method:

(1) Six RAGE antigen protein extracellular domains (ECD) from different sources and with different tags were expressed and synthesized. The antigen design information and the results for expression and synthesis are shown in Table 1 below, wherein the amino acid sequence of Human-RAGE-ECD-Fc (IgG1) is represented by SEQ ID NO: 13 (AQNITARIGEPLVLKCKGAPKKPPQRLEWKLNTGRTEAWKVLSPQGGGPWDSVARVLP NGSLFLPAVGIQDEGIFRCQAMNRNGKETKSNYRVRVYQIPGKPEIVDSASELTAGVPNK VGTCVSEGSYPAGTLSWHLDGKPLVPNEKGVSVKEQTRRHPETGLFTLQSELMVTPARG GDPRPTFSCSFSPGLPRHRALRTAPIQPRVWEPVPLEEVQLVVEPEGGAVAPGGTVTLTC EVPAQPSPQIHWMKDGVPLPLPPSPVLILPEIGPQDQGTYSCVATHSSHGPQESRAVSISII EPGEEGPTAGSVGGSGLGTLAEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQD WLNGKEYKCKVSNKALPAPIEKTISKAKGQPREPQVYTLPPSRDELTKNQVSLTCLVKG FYPSDIAVEWESNGQPENNYKTTPPVLDSDGSFFLYSKLTVDKSRWQQGNVFSCSVMHE ALHNHYTQKSLSLSPGK), the amino acid sequence of Cyno-RAGE-ECD-Fc (IgG1) is represented by SEQ ID NO 14 (AQNITARIGEPLVLKCKGAPKKPPQQLEWKLNTGRTEAWKVLSPQGGPWDSVARVLPN GSLFLPAVGIQDEGIFRCQAMNRNGKETKSNYRVRVYQIPGKPEIIDSASELTAGVPNKV GTCVSEGSYPAGTLSWHLDGKPLVPNEKGVSVKEETRRHPETGLFTLQSELMVTPARGG NPHPTFSCSFSPGLPRRRALHTAPIQPRVWEPVPLEEVQLVVEPEGGAVAPGGTVTLTCE VPAQPSPQIHWMKDGVPLPLSPSPVLILPEIGPQDQGTYRCVATHPSHGPQESRAVSISIIE PGEEGPTAGSVGGSGPGTLAEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDW LNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEAL HNHYTQKSLSLSPGK), the amino acid sequence of Mouse-RAGE-ECD-Fc (IgG1) is represented by SEQ ID NO 15 (GQNITARIGEPLVLSCKGAPKKPPQQLEWKLNTGRTEAWKVLSPQGGPWDSVARILPN GSLLLPATGIVDEGTFRCRATNRRGKEVKSNYRVRVYQIPGKPEIVDPASELTASVPNKV0 GTCVSEGSYPAGTLSWHLDGKLLIPDGKETLVKEETRRHPETGLFTLRSELTVIPTQGGT HPTFSCSFSLGLPRRRPLNTAPIQLRVREPGPPEGIQLLVEPEGGIVAPGGTVTLTCAISAQ PPPQVHWIKDGAPLPLAPSPVLLLPEVGHEDEGTYSCVATHPSHGPQESPPVSIRVTETGD EGPAEGSVGESGLGTLAEPKSCDKTHTCPPCPAPELLGGPSVFLFPPKPKDTLMISRTPEV TCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSRDELTKNQVSLTCLVKGFYPS DIAVEWESNGQPENNYKTTPPVLDSDGSFFLYSKLTVDKSRWQQGNVFSCSVMHEALH NHYTQKSLSLSPGK), the amino acid sequence of Human-RAGE-ECD-His is represented by SEQ ID NO: 16 (AQNITARIGEPLVLKCKGAPKKPPQRLEWKLNTGRTEAWKVLSPQGGGPWDSVARVLP NGSLFLPAVGIQDEGIFRCQAMNRNGKETKSNYRVRVYQIPGKPEIVDSASELTAGVPNK VGTCVSEGSYPAGTLSWHLDGKPLVPNEKGVSVKEQTRRHPETGLFTLQSELMVTPARG GDPRPTFSCSFSPGLPRHRALRTAPIQPRVWEPVPLEEVQLVVEPEGGAVAPGGTVTLTC EVPAQPSPQIHWMKDGVPLPLPPSPVLILPEIGPQDQGTYSCVATHSSHGPQESRAVSISII EPGEEGPTAGSVGGSGLGTLAGGSGGSHHHHHHHH), the amino acid sequence of Cyno-RAGE-ECD-His is represented by SEQ ID NO: 17 (AQNITARIGEPLVLKCKGAPKKPPQQLEWKLNTGRTEAWKVLSPQGGPWDSVARVLPN GSLFLPAVGIQDEGIFRCQAMNRNGKETKSNYRVRVYQIPGKPEIIDSASELTAGVPNKV GTCVSEGSYPAGTLSWHLDGKPLVPNEKGVSVKEETRRHPETGLFTLQSELMVTPARGG NPHPTFSCSFSPGLPRRRALHTAPIQPRVWEPVPLEEVQLVVEPEGGAVAPGGTVTLTCE VPAQPSPQIHWMKDGVPLPLSPSPVLILPEIGPQDQGTYRCVATHPSHGPQESRAVSISIIE PGEEGPTAGSVGGSGPGTLAGGSGGSHHHHHHHH), and the amino acid sequence of Mouse-RAGE-ECD-His is represented by SEQ ID NO: 18 (GQNITARIGEPLVLSCKGAPKKPPQQLEWKLNTGRTEAWKVLSPQGGPWDSVARILPN GSLLLPATGIVDEGTFRCRATNRRGKEVKSNYRVRVYQIPGKPEIVDPASELTASVPNKV GTCVSEGSYPAGTLSWHLDGKLLIPDGKETLVKEETRRHPETGLFTLRSELTVIPTQGGT HPTFSCSFSLGLPRRRPLNTAPIQLRVREPGPPEGIQLLVEPEGGIVAPGGTVTLTCAISAQ PPPQVHWIKDGAPLPLAPSPVLLLPEVGHEDEGTYSCVATHPSHGPQESPPVSIRVTETGD EGPAEGSVGESGLGTLAGGSGGSHHHHHHHH).

TABLE 1

| | | | | Relative | | Total |
|---|---|---|---|---|---|---|
| | | | Protein | molecular | Concentration | mass |
| Name of protein | Species/Tag | Type | No. | mass (kDa) | (mg/mL) | (mg) |
| Human-RAGE-ECD-Fc(IgG1) | Human, Fc | Antigen | P38406 | 120.12 | 4.21 mg/ml | 17.66 |
| Cyno-RAGE-ECD-Fc(IgG1) | Monkey, Fc | Antigen | P38408 | 120.04 | 3.78 mg/ml | 12.26 |
| Mouse-RAGE-ECD-Fc(IgG1) | Mouse, Fc | Antigen | P38410 | 119.66 | 3.24 mg/ml | 6.26 |
| Human-RAGE-ECD-His | Human, His | Antigen | P38405 | 35.48 | 4.12 mg/ml | 24.26 |
| Cyno-RAGE-ECD-His | Monkey, His | Antigen | P38407 | 35.45 | 3.07 mg/ml | 17.66 |
| Mouse-RAGE-ECD-His | Mouse, His | Antigen | P38409 | 35.26 | 4.71 mg/ml | 21.91 |

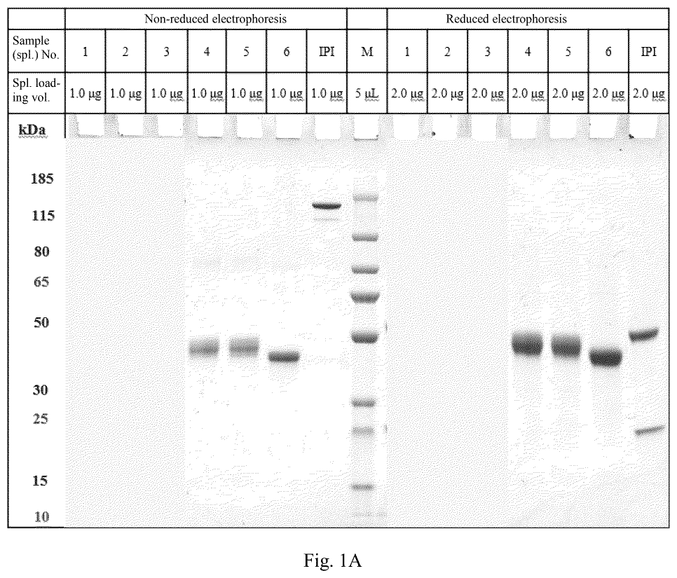

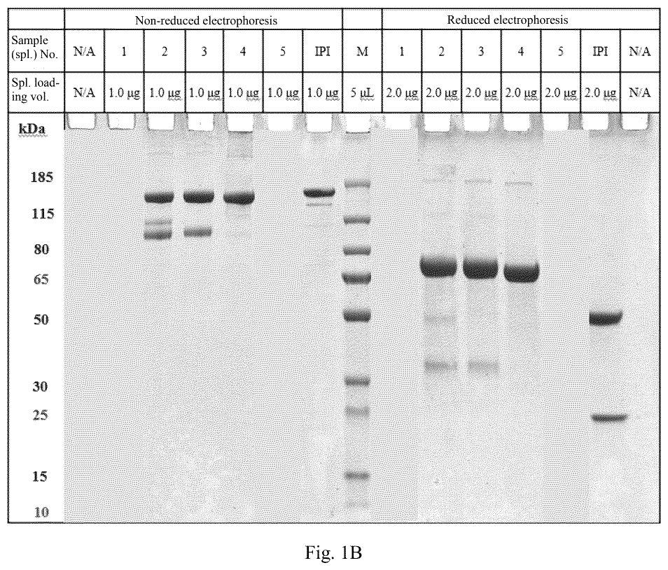

(2) The six RAGE antigens were subjected to SDS-PAGE-Coomassie Brilliant Blue staining. The results are shown in FIGS. 1A-1B. The purity was estimated by the grayscale of the bands. The results are shown in Tables 2-3 below:

TABLE 2

| Sample | | | | | |
|---|---|---|---|---|---|
| No. in | | Protein | Protein | Mw | Reduced |
| FIG. 1A | Name of protein | No. | type | (kDa) | purity (%) |
| 4 | Human-RAGE-ECD-His | P38405 | Antigen | 35.48 | >85.0 |
| 5 | Cyno-RAGE-ECD-His | P38407 | Antigen | 35.45 | >85.0 |
| 6 | Mouse-RAGE-ECD-His | P38409 | Antigen | 35.26 | >85.0 |

TABLE 3

| Sample | | | | | |
|---|---|---|---|---|---|
| No. in | | Protein | Protein | Mw | Reduced |
| FIG. 1B | Name of protein | No. | type | (kDa) | purity (%) |
| 2 | Human-RAGE-ECD- | P38406 | Antigen | 120.12 | 77.0 |
| | Fc(IgG1) | | | | |
| 3 | Cyno-RAGE-ECD- | P38408 | Antigen | 120.04 | 83.7 |
| | Fc(IgG1) | | | | |
| 4 | Mouse-RAGE-ECD- | P38410 | Antigen | 119.66 | 97.0 |
| | Fc(IgG1) | | | | |

Experimental results: all six RAGE antigens were successfully expressed, and the purity of all of them was over 70% in reduced SDS-PAGE.

### 1.2 Detection of Anti-RAGE Antibody Expression is Used to Evaluate Antibody Activity, so as to Take the Antibodies as the Control of the Screened Antibodies.

### Experimental Method:

(1) Two control anti-RAGE antibodies were expressed and synthesized. The relevant information, and expression and synthesis results are shown in Table 4 below. Particularly, the antibody h11E6.16 may be synthesized according to the method in Patent ES2577718T3. The amino acid sequence of the heavy chain of the antibody h11E6.16 is represented by SEQ ID NO: 19 (EIQLVQSGAEVKKPGASVKVSCKASGYTFTNFGMNWVRQAPGQGLEWMGYINTNTGE SIYSEEFKGRFTFTLDTSTSTAYMELSSLRSEDTAVYFCARSRMVTAYGMDYWGQGTSV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSRDELTKNQVSLTCLVKGFYPSDIAVEWESNGQPENNYKTTPPVLDSDGSFFLYSKLT VDKSRWQQGNVFSCSVMHEALHNHYTQKSLSLSPGK), and the light chain is represented by SEQ ID NO 20 (EIVMTQSPATLSLSPGERATLSCKASQNVGTAVAWYQQKPGQSPRLLIFSASNRYTGVP ARFSGSGSGTDFTLTISSLQSEDFAVYFCQQYSSYPLTFGQGTKLEIKRTVAAPSVFIFPPS DEQLKSGTASVVCLLNNFYPREAKVQWKVDNALQSGNSQESVTEQDSKDSTYSLSSTLT LSKADYEKHKVYACEVTHQGLSSPVTKSFNRGEC).

TABLE 4

| | | | | Relative | | | Total |
|---|---|---|---|---|---|---|---|
| | Origin of | | Protein | molecular | Concentration | | mass |
| Name of protein | species | Type | No. | mass (kDa) | (mg/mL) | | (mg) |
| Anti-hRAGE | Mouse | Antibody | P46085 | 120.12 | 0.5 | mg/ml | 0.92 |
| (R&D systems/ | | | | | | | |
| MAB11451) | | | | | | | |
| h11E6.16 | Human | Antibody | P38411 | 145.18 | 1.77 | mg/ml | 0.78 |

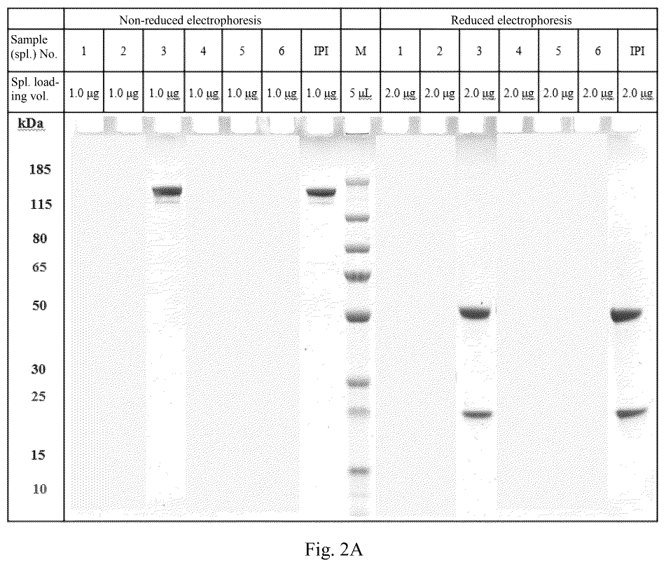

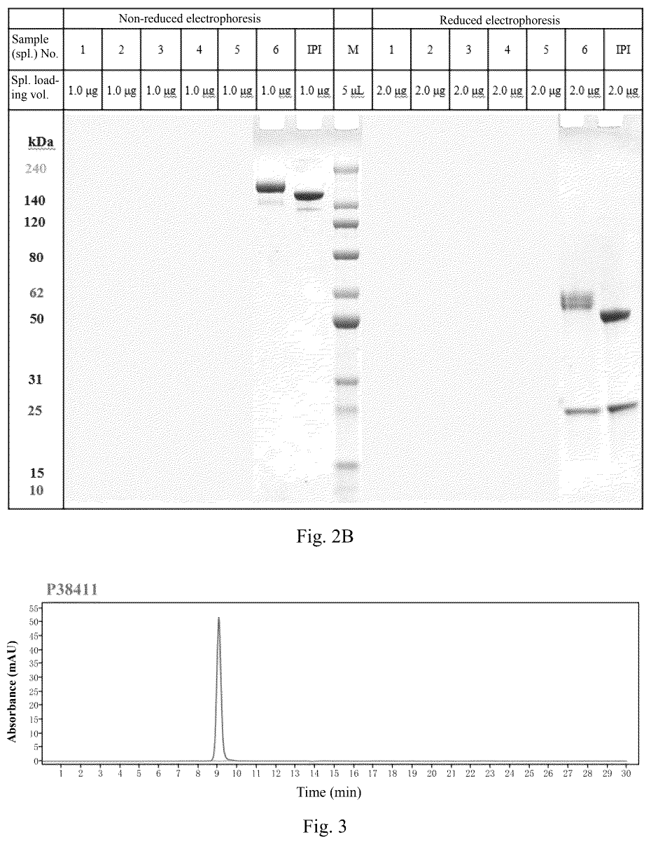

(2) Two control anti-RAGE antibodies were subjected to SDS-PAGE-Coomassie Brilliant Blue staining. The experimental results are shown in FIGS. 2A-2B. The purity was estimated by the grayscale of the bands. The results are shown in Tables 5-6 below:

TABLE 5

| Sample | | | | | |
|---|---|---|---|---|---|
| No. in | | Protein | | Mw | Reduced |
| FIG. 2A | Name of protein | No. | Protein type | (kDa) | purity (%) |
| 3 | h11E6.16 | P38411 | Full length | 145.18 | >95.0 |
| | | | antibody | | |

TABLE 6

| Sample | | | | | |
|---|---|---|---|---|---|
| No. in | | Protein | | Mw | Reduced |
| FIG. 2B | Name of protein | No. | Protein type | (kDa) | purity (%) |
| 6 | Anti-hRAGE | P46085 | Full length | NA | >95.0 |
| | | | antibody | | |

(3) The h11E6.16 antibody was subjected to HPLC-SEC detection, and the results are shown in Table 7 and FIG. 3.

TABLE 7

| | Retention time | High molecular | | Low molecular |
|---|---|---|---|---|
| Protein | of monomer | weight polymer | Monomer | weight |
| No. | (min) | (%) | (%) | substance (%) |
| BSA | 10.20 | 9.02 | 87.64 | 3.34 |
| Herceptin | 9.21 | 1.45 | 98.55 | / |
| P38411 | 9.06 | / | 100.00 | / |

Experimental results: h11E6.16 antibody was successfully expressed, with a monomer rate of 100%.

1.3 Detecting the Binding Activity of Different Antigens with h11E6.16 Antibody (P38411).

### Experimental Method:

(1) Elisa plates were coated with different antigens (the six RAGE antigens synthesized in Example 1, ipilimumab (Sanyou Bio/CHA032) (negative control), and 1% skim milk as blank control), wherein the antigens were diluted with PBS to a working concentration of 2 μg/mL, and L was added to each well to incubate at 4° C. overnight.

(2) The plates were washed three times with PBST (PBS supplemented with 0.05% TWEEN-20), and then blocked with 5% skim milk (prepared with PBS) at room temperature for 2 h.

(3) The plates were washed three times with PBST, then adding h11E6.16 (P38411) diluted with 1% skim milk (prepared with PBS) at a volume of 30 μL per well to incubate at room temperature for 60 min.

(4) The plates were washed three times with PBST, then adding goat-anti-human-κ+λ-HRP (Millipore/AP502P, AP506P) diluted with 1% skim milk (dilution ratio 1:4000) to incubate at room temperature for 50 min.

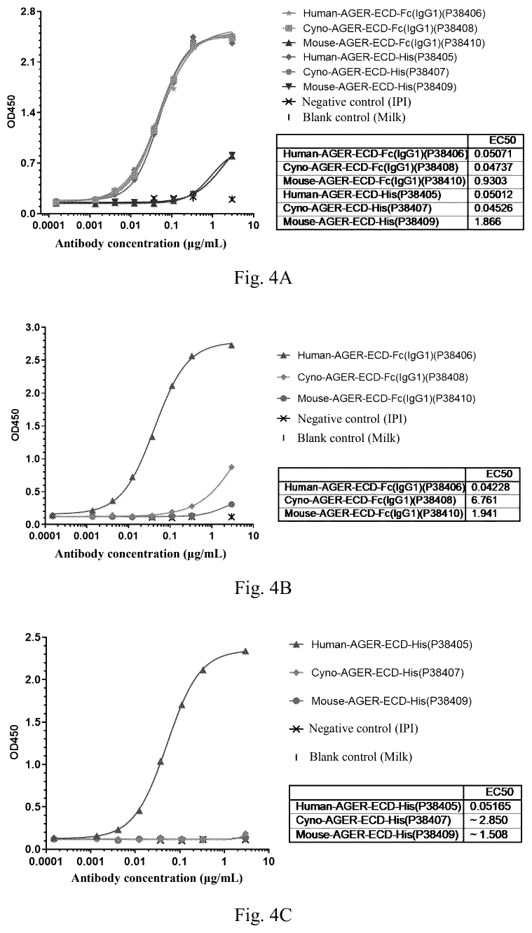

(5) Tetramethylbenzidine (TMB, HRP chromogenic substrate) was added, the reaction was terminated with 2 M HRP reaction termination solution, and OD450 was read. The results are shown in FIG. 4A.

Experimental results: h11E6.16 antibody (P38411) has better binding ability with human and monkey antigens, but weaker binding ability with mouse antigens.

1.4 Detecting the Binding Activity of Different Antigens with Anti-hRAGE (P46085) (Cat. No.: R&D Systems/MAB11451).

### Experimental Method:

(1) Elisa plates were coated with different antigens, wherein the antigens were diluted with PBS to a working concentration of 4 μg/mL, and 30 μL was added to each well to incubate at 4° C. overnight.

(2) The plates were washed three times with PBST, and then blocked with 5% skim milk (prepared with PBS) at room temperature for 2 h.

(3) The plates were washed three times with PBST, then adding 30 μL of Anti-hRAGE (P46085) diluted with 1% skim milk (prepared with PBS) to each well to incubate at room temperature for 60 min.

(4) The plates were washed three times with PBST, then adding goat-anti-mouse-IgG (1+2a+2b+3)-HRP (Jackson/115-035-164) diluted with 1% skim milk (dilution ratio 1:4000) to incubate at room temperature for 50 min.

(5) TMB was added, the reaction was terminated with reaction termination solution, and OD450 was read. The results are shown in FIGS. 4B-4C.

Experimental results: Anti-hRAGE (P46085) has a strong binding ability to human antigens, but a very weak binding ability to monkey and mouse antigens.

### 1.5 Construction of Biotin-Labeled RAGE Antigen.

### Experimental Method:

(1) Antigen proteins were labeled with biotin N-hydroxysulfosuccinimide (NHS) ester (Thermo Scientific/20217).

(2) The biotin-labeled antigens were diluted with PBS to coat the Elisa plate, and 30 μL was added to each well to incubate at 4° C. overnight.

(3) The plates were washed three times with PBST, and then blocked with 5% skim milk (prepared with PBS) at room temperature for 2 h.

(4) The plates were washed three times with PBST, and NeutrAvidin-HRP (ThermoFisher/31001) diluted with 1% skim milk (dilution ratio 1:2000) was added to incubate at room temperature for 60 min.

(5) The plates were washed 12 times with PBST.

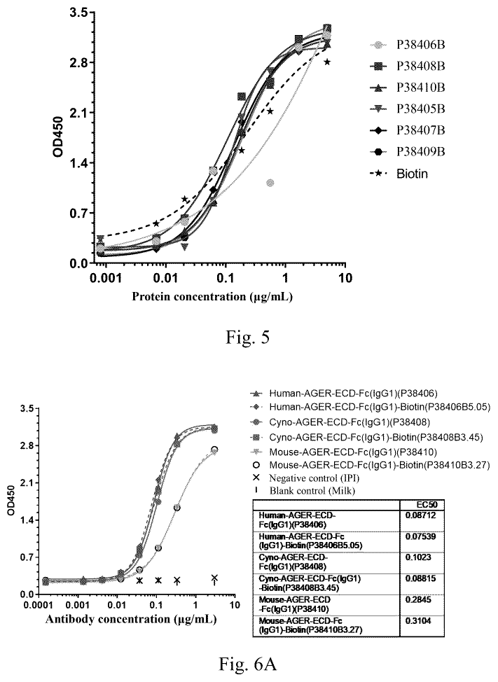

(6) 30 μL of TMB was added to each well for color development, then the termination solution was added to terminate the reaction, and the OD450 was read. The results are shown in FIG. 5.

Experimental results: All antigen proteins were successfully labeled with biotin.

1.6 Verifying that Biotin Labeling does not Affect the Activity of Antigen Binding Antibody.

### Experimental Method:

(1) Elisa plates were coated with the above biotin N-hydroxysulfosuccinimide (NHS) ester-labeled antigens, wherein the antigens were diluted with PBS to a working concentration of 4 μg/mL, and 30 μL was added to each well to incubate at 4° C. overnight.

(2) The plates were washed three times with PBST, and then blocked with 5% skim milk (prepared with PBS) at room temperature for 2 h.

(3) The plates were washed three times with PBST, and h11E6.16 (P38411) diluted with 1% skim milk (prepared with PBS) was added at a volume of 30 μL per well to incubate at room temperature for 60 min.

(4) The plates were washed three times with PBST, and goat-anti-human-κ+λ-RP (Millipore/AP502P, AP506P) diluted with 1% skim milk (dilution ratio 1:4000) was added to incubate at room temperature for 50 min; alternatively, goat-anti-human-IgG-Fc-HRP (abeam, ab97225) diluted with 1% skim milk (dilution ratio 1:8000) was added to incubate at room temperature for 50 min.

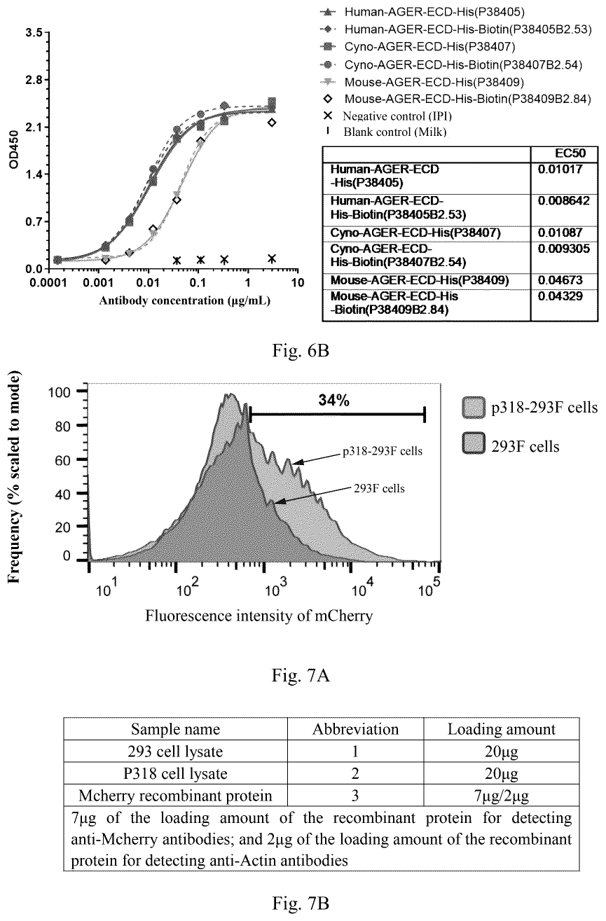

(5) TMB was added, the reaction was terminated with termination solution, and OD450 was read. The results are shown in FIGS. 6A-6B.

Experimental results: before and after biotin labeling, the RAGE antigens from different sources have comparable activities in binding antibodies.

1.7 A Cell Line Highly Expressing RAGE Antigen Proteins was Constructed by Stably Transfecting 293F Suspension Cells with a Plasmid.

### Experimental Method:

(1) Plasmid p318 (RAGE(1-404)-3×flag-mCherry) was constructed, and its sequence is represented by SEQ ID NO: 21 (MAAGTAVGAWVLVLSLWGAVVGAQNITARIGEPLVLKCKGAPKKPPQRLEWKLNTG RTEAWKVLSPQGGGPWDSVARVLPNGSLFLPAVGIQDEGIFRCQAMNRNGKETKSNYR VRVYQIPGKPEIVDSASELTAGVPNKVGTCVSEGSYPAGTLSWHLDGKPLVPNEKGVSV KEQTRRHPETGLFTLQSELMVTPARGGDPRPTFSCSFSPGLPRHRALRTAPIQPRVWEPVP LEEVQLVVEPEGGAVAPGGTVTLTCEVPAQPSPQIHWMKDGVPLPLPPSPVLILPEIGPQ DQGTYSCVATHSSHGPQESRAVSISIIEPGEEGPTAGSVGGSGLGTLALALGILGGLGTAA LLIGVILWQRRQRRGEERKAPENQEEEEERAELNQSEEPEAGESSTGGPDYKDHDGDYK DHDIDYKDDDDKMVSKGEEDNMAIIKEFMRFKVHMEGSVNGHEFEIEGEGEGRPYEGT QTAKLKVTKGGPLPFAWDILSPQFMYGSKAYVKHPADIPDYLKLSFPEGFKWERVMNF EDGGVVTVTQDSSLQDGEFIYKVKLRGTNFPSDGPVMQKKTMGWEASSERMYPEDGA LKGEIKQRLKLKDGGHYDAEVKTTYKAKKPVQLPGAYNVNIKLDITSHNEDYTIVEQYE RAEGRHSTGGMDELYK).

(2) The above plasmid was transiently transfect into 293F suspension cells by using chemical transfection reagent, then collecting the cells for later use.

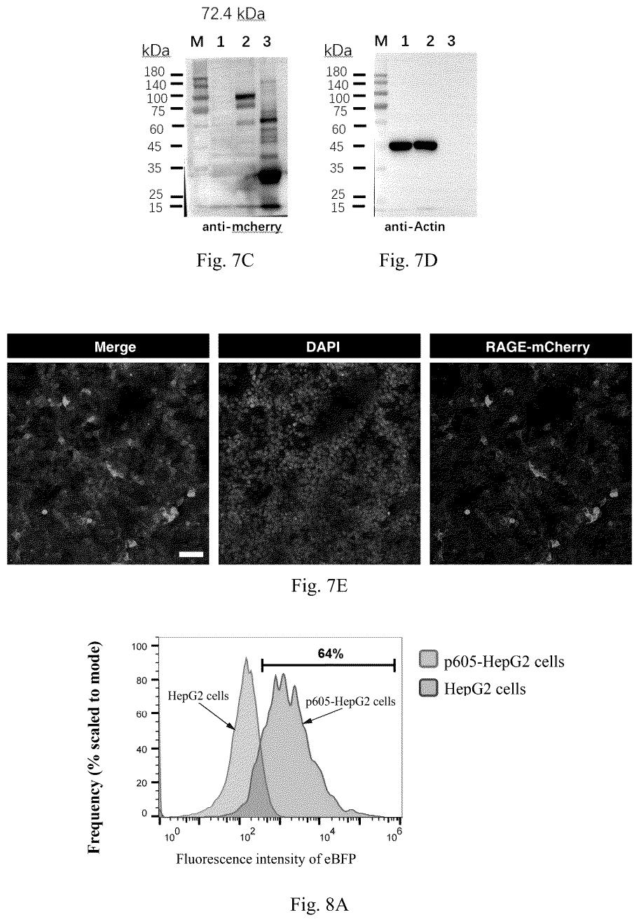

(3) The cells collected in step (2) were used for detecting mCherry fluorescence by Attune™ NxT flow cytometer (Thermo Fisher, A24860). The experimental results are shown in FIG. 7A. The experimental results show that p318-transiently transfected 293F cells highly expressed RAGE-3×flag-mCherry.

(4) The cells collected in step (2) were lysed for detecting conjugated mCherry fluorescent protein by Western blotting (WB). The experimental results are shown in FIGS. 7B-7D. The experimental results show that p318-transiently transfected 293F cells highly expressed RAGE-3×flag-mCherry.

(5) 0.1 mg/mL poly-lysine (PDL) was added to the confocal culture dish to place in an incubator (37° C., 5% CO₂) for coating for 30 min. The dish was then washed three times with ultrapure water, and the cells collected in step (2) were added to incubate overnight in an incubator so as to allow the cells to adhere to the wall. 4% PFA was added to the dish to fix the cells, and the cells were washed with PBS. Then 0.5 μg/mL DAPI dye was added to stain at room temperature for 30 min. The dish was washed with PBS, and 70% glycerol (prepared with PBS) was added to transfer the dish to a confocal microscope for observation and photography. The photograph is shown in FIG. 7E. The experimental results show that p318-transiently transfected 293F cells highly expressed RAGE-3×flag-mCherry (scale bar: 100 m).

1.8 A Cell Line Highly Expressing RAGE Antigens was Constructed by Transiently Transfecting HepG2 Adherent Cells with a Plasmid.

### Experimental Method:

(1) Plasmid p605 (RAGE(1-404)-3×flag-eBFP) was construct, and its sequence is represented by SEQ ID NO: 22 (MAAGTAVGAWVLVLSLWGAVVGAQNITARIGEPLVLKCKGAPKKPPQRLEWKLNTG RTEAWKVLSPQGGGPWDSVARVLPNGSLFLPAVGIQDEGIFRCQAMNRNGKETKSNYR VRVYQIPGKPEIVDSASELTAGVPNKVGTCVSEGSYPAGTLSWHLDGKPLVPNEKGVSV KEQTRRHPETGLFTLQSELMVTPARGGDPRPTFSCSFSPGLPRHRALRTAPIQPRVWEPVP LEEVQLVVEPEGGAVAPGGTVTLTCEVPAQPSPQIHWMKDGVPLPLPPSPVLILPEIGPQ DQGTYSCVATHSSHGPQESRAVSISIIEPGEEGPTAGSVGGSGLGTLALALGILGGLGTAA LLIGVILWQRRQRRGEERKAPENQEEEEERAELNQSEEPEAGESSTGGPDYKDHDGDYK DHDIDYKDDDDKMVSKGEELFTGVVPILVELDGDVNGHKFSVSGEGEGDATYGKLTLK FICTTGKLPVPWPTLVTTLSHGVQCFSRYPDHMKQHDFFKSAMPEGYVQERTIFFKDDG NYKTRAEVKFEGDTLVNRIELKGIDFKEDGNILGHKLEYNFNSHNVYIMADKQKNGIKA NFKIRHNIEDGSVQLADHYQQNTPIGDGPVLLPDSHYLSTQSALSKDPNEKRDHMVLLEF VTAAGITLGMDELYK) is shown.

(2) The above plasmid was transiently transfect into HepG2 cells by using chemical transfection reagent, then collecting the cells for later use.

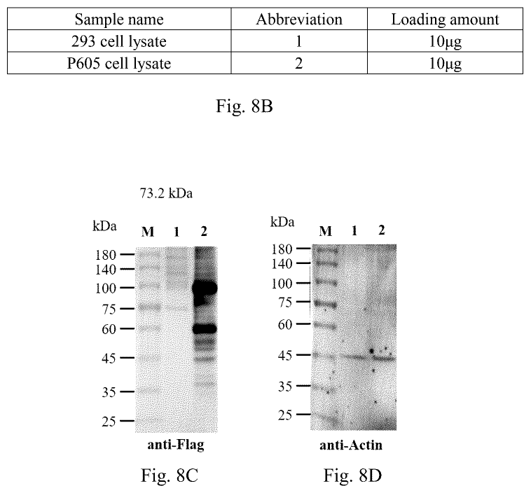

(3) The cells collected in step (2) were used for detecting eBFP fluorescence by flow cytometry. The experimental results are shown in FIG. 8A. The experimental results show that p605-transiently transfected HepG2 cells highly expressed RAGE-3×flag-eBFP.

(4) The cells collected in step (2) were lysed for detecting conjugated flag tag by Western blotting. The experimental results are shown in FIGS. 8B-8D. The experimental results show that p605-transiently transfected HepG2 cells highly expressed RAGE-3×flag-eBFP.

### Example 2: Antibody Library Screening

### 2.1 Phage Antibody Library Screening

### Experimental Method:

Three rounds of screening were performed on the phage antibody libraries (as shown in Tables 8 and 9 below) with antigen proteins conjugated with different labels from different sources, to screen out antibodies that specifically bind to human, mouse and monkey antigens, as well as antigen positive cells.

TABLE 8

| Round | Protein | | | | | | | | |
|---|---|---|---|---|---|---|---|---|---|
| No. | amount | Input: NL | | | | | | | |
| First | 100 | Human-RAGE-ECD-Fc | | | | Human-RAGE-ECD-Fc-Bio | | | |
| panning | μg/mL | | | | | | | | |
| Second | 30 | Human- | Human- | Cyno- | Cell | Human- | Human- | Cyno- | Cell |
| panning | μg/mL | RAGE- | RAGE- | RAGE- | | RAGE- | RAGE- | RAGE- | |
| | | ECD-Fc | ECD- | ECD-His | | ECD- | ECD-His | ECD-His | |
| | | | His-Bio | | | Fc- Bio | | | |
| Third | 10 | Human- | Human- | Mouse- | Human- | Human- | Human- | Mouse- | Human- |
| panning | μg/mL | RAGE- | RAGE- | RAGE- | RAGE- | RAGE- | RAGE- | RAGE- | RAGE- |
| | | ECD-Fc | ECD-Fc | ECD- His | ECD-Fc | ECD- | ECD- | ECD- His | ECD- |
| | | | | | | Fc- Bio | Fc- Bio | | Fc-Bio |

TABLE 9

| Round | Protein | | | | | | | | |
|---|---|---|---|---|---|---|---|---|---|
| No. | amount | Input: NL | | | | | | | |
| First | 100 | Human-RAGE-ECD-His | | | | Human-RAGE-ECD-His-Bio | | | |
| panning | μg/mL | | | | | | | | |
| Second | 30 | Human- | Human- | Cyno- | Cell | Human- | Human- | Cyno- | Cell |
| panning | μg/mL | RAGE- | RAGE- | RAGE- | | RAGE- | RAGE- | RAGE- | |
| | | ECD-His | ECD- | ECD- His | | ECD- | ECD-Fc | ECD-His | |
| | | | Fc-Bio | | | His-Bio | | | |
| Third | 10 | Human- | Human- | Mouse- | Human- | Human- | Human- | Mouse- | Human- |
| panning | μg/mL | RAGE- | RAGE- | RAGE- | RAGE- | RAGE- | RAGE- | RAGE- | RAGE- |
| | | ECD-His | ECD-His | ECD-His | ECD- His | ECD- | ECD- | ECD-His | ECD- |
| | | | | | | His-Bio | His-Bio | | His-Bio |

### 2.2 Detection of the Specific Binding Ability of Candidate Phage Antibody Pools to Cells Expressing Human RAGE Antigens

### Experimental Method:

(1) According to the screening strategy in section “2.1 Phage antibody library screening”, the p318-293F cells with high expression of RAGE antigens constructed in section “1.7 a cell line highly expressing RAGE antigen proteins was constructed by stably transfecting 293F suspension cells with a plasmid” and 293F cells (blank cells) were used for respectively plating at 1E5 cells/well, and the cells were washed with FACS buffer.

(2) 100 μL of antibody dilution was added to the cells to incubate at 4° C. for 60 min, then centrifuging, and washing the cells twice with FACS buffer.

(3) 100 μL of M13 (Sino Biological 11973-MM05T-P 1:500), or anti-human Fc (PE) (abcam ab98596 1:300), or anti-Mouse Fc (abcam 98742 1:300) was added to the cells to incubate at 4° C. for 30 min.

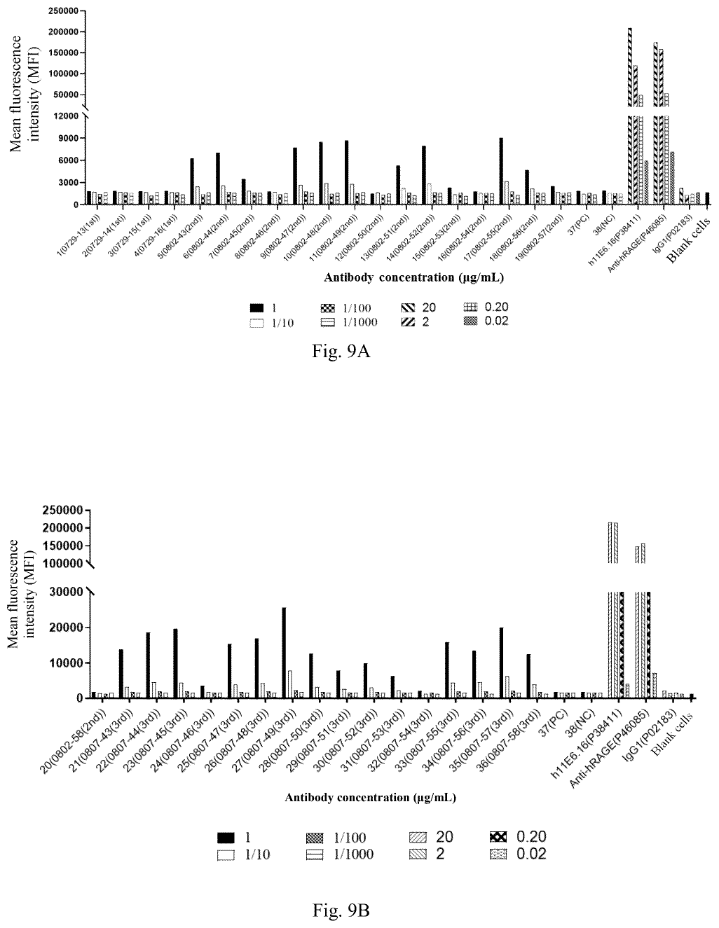

(4) The cells were resuspended in FACS buffer, and detected by flow cytometry. The results of mean fluorescence intensity (MFI) of cells which show the specific binding ability of each candidate phage antibody pools to p318-293F cells expressing human RAGE antigens are shown in FIGS. 9A-9B, and the results of the mean fluorescence intensity of cells which show the specific binding ability of each candidate phage antibody pools to 293F cells are shown in Tables 10-11, wherein PC represents positive bacteria infected with phages, and NC represents negative bacteria without presence of phages.

TABLE 10

| | Tested samples | | | | | | | | | |
|---|---|---|---|---|---|---|---|---|---|---|
| | 1(0729- | 2(0729- | 3(0729- | 4(0729- | 5(0802- | 6(0802- | 7(0802- | 8(0802- | 9(0802- | 10(0802- |
| | 13(1st)) | 14(1st)) | 15(1st)) | 16(1st)) | 43(2nd)) | 44(2nd)) | 45(2nd)) | 46(2nd)) | 47(2nd)) | 48(2nd)) |
| MFI | 580.8 | 610 | 606.7 | 628.6 | 596.2 | 631.3 | 621.8 | 599.9 | 637.4 | 530.2 |

| | Tested samples | | | | | | | |
|---|---|---|---|---|---|---|---|---|
| | 11(0802- | 12(0802- | 13(0802- | 14(0802- | 15(0802- | 16(0802- | 17(0802- | 18(0802- |
| | 49(2nd)) | 50(2nd)) | 51(2nd)) | 52(2nd)) | 53(2nd)) | 54(2nd)) | 55(2nd)) | 56(2nd)) |
| MFI | 618.3 | 593 | 637.4 | 625.6 | 616.2 | 625.4 | 626.1 | 607.4 |

| | Tested samples | | | | | | |
|---|---|---|---|---|---|---|---|
| | 19(0802- | | | | Anti- | | cell |
| | 57(2nd)) | 37(PC) | 38(NC) | h11E6.16(P38411) | hRAGE(P46085) | IgG1(P02183) | only |
| MFI | 610.2 | 614.5 | 664.8 | 533.1 | 545.5 | 949.6 | 517.8 |

TABLE 11

| | Tested samples | | | | | | | | | |
|---|---|---|---|---|---|---|---|---|---|---|
| | 20(0802- | 21(0807- | 22(0807- | 23(0807- | 24(0807- | 25(0807- | 26(0807- | 27(0807- | 28(0807- | 29(0807- |
| | 58(2nd)) | 43(3rd)) | 44(3rd)) | 45(3rd)) | 46(3rd)) | 47(3rd)) | 48(3rd)) | 49(3rd)) | 50(3rd)) | 51(3rd)) |
| MFI | 684.6 | 700.8 | 643.1 | 707.2 | 633.3 | 695 | 661.3 | 624 | 685.1 | 594.8 |

| | Tested samples | | | | | | |
|---|---|---|---|---|---|---|---|
| | 30(0807- | 31(0807- | 32(0807- | 33(0807- | 34(0807- | 35(0807- | 36(0807- |
| | 52(3rd)) | 53(3rd)) | 54(3rd)) | 55(3rd)) | 56(3rd)) | 57(3rd)) | 58(3rd)) |
| MFI | 699.1 | 530 | 665.7 | 662.4 | 667.2 | 651.5 | 622.3 |

### Analysis of the Experimental Results:

(1) As shown in FIGS. 9A-9B, phage libraries 5 (0802-43 (2nd)), 6 (0802-44 (2nd)), 7 (0802-45 (2nd)), 9 (0802-47 (2nd)), 10 (0802-48 (2nd)), 11 (0802-49 (2nd)), 13 (0802-51 (2nd)), 14 (0802-52 (2nd)), 17 (0802-55 (2nd)), 18 (0802-56 (2nd)) (0807-43 (3rd)), 22 (0807-44 (3rd)), 23 (0807-45 (3rd)), 24 (0807-46 (3rd)), 25(0807-47(3rd)), 26(0807-48(3rd)), 27(0807-49(3rd)), 28(0807-50(3rd)), 29(0807-51(3rd)), 30(0807-52(3rd)), 31(0807-53(3rd)), 33(0807-55(3rd)), 34(0807-56(3rd)), 35(0807-57(3rd)), and 36(0807-58(3rd)) have better binding ability to p318-293F cells.

(2) It can be seen from Tables 10-11, all phages fail to bind to 293F cells.

2.3 Detecting the Specific Binding Ability of Candidate Phage Antibody Pools to RAGE Antigens of Different Species, and Screening Pools with Cross-Reactivity Among Human, Mouse and Monkey.

### Experimental Method (See Sections 1.3 and 1.4 of Example 1 for the Methods):

(1) Elisa plates were coated with different antigens human-RAGE-ECD-His (P38405), cyno-RAGE-ECD-His (P38407), mouse-RAGE-ECD-His (P38409) and PBS (blank control), wherein the antigen was diluted with PBS to a working concentration of 4 μg/mL, and 30 μL was added to each well to incubate at 4° C. overnight.

(2) The plates were washed three times with PBST, and then blocked with 5% skim milk (prepared with PBS) at room temperature for 2 h.

(3) The plates were washed three times with PBST, and 30 μL of phage antibody pool or control phage antibody (H11E6.16) and blank control (NC) were added to each well to incubate at room temperature for 60 min.

(4) The plates were washed three times with PBST, then adding mouse Anti-M13 (Sinobiological/11973-MM05T-H) diluted with 1% skim milk (dilution ratio 1:8000) to incubate at room temperature for 50 min.

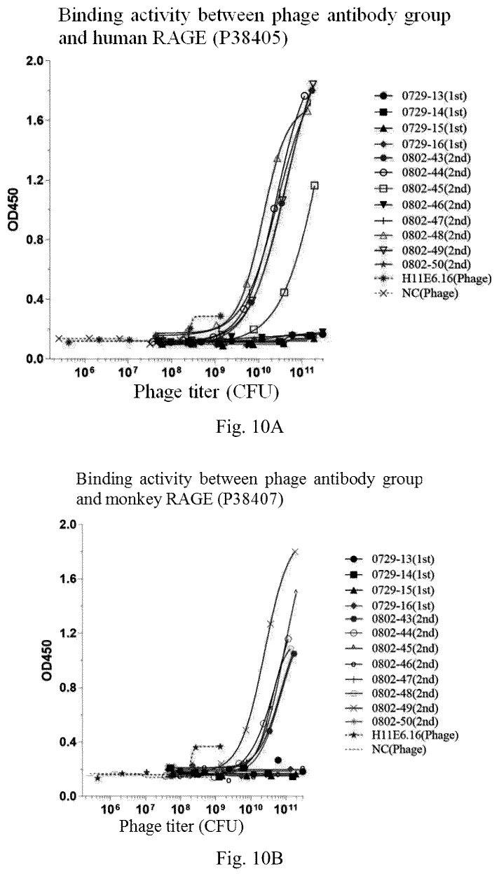

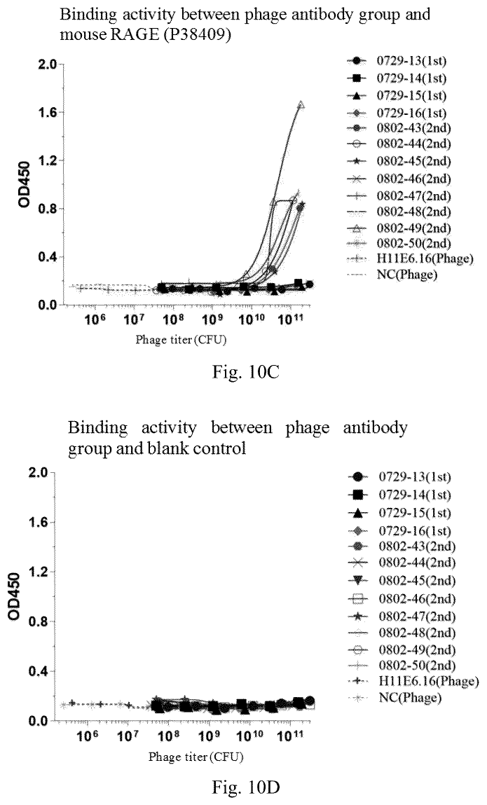

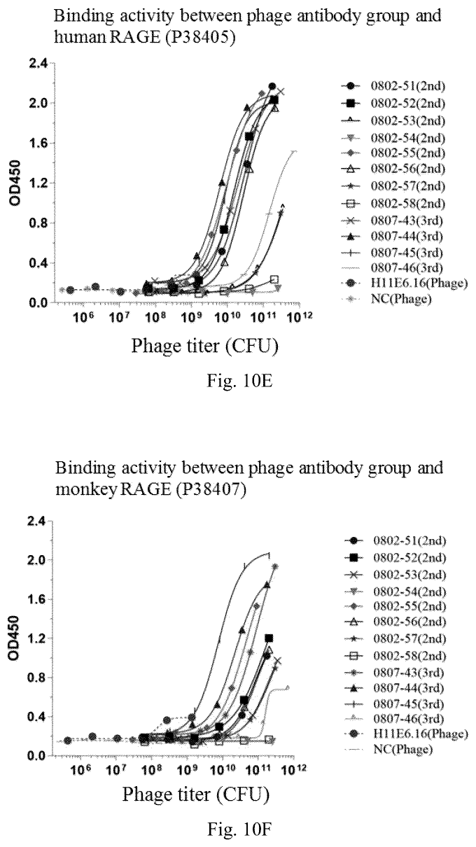

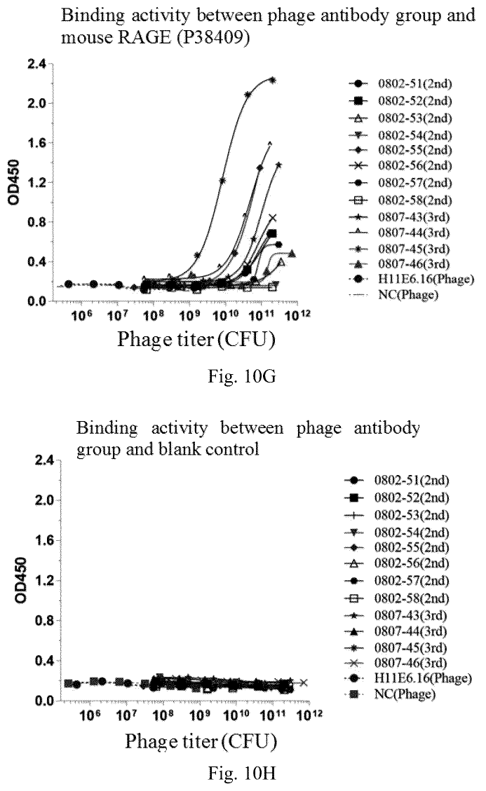

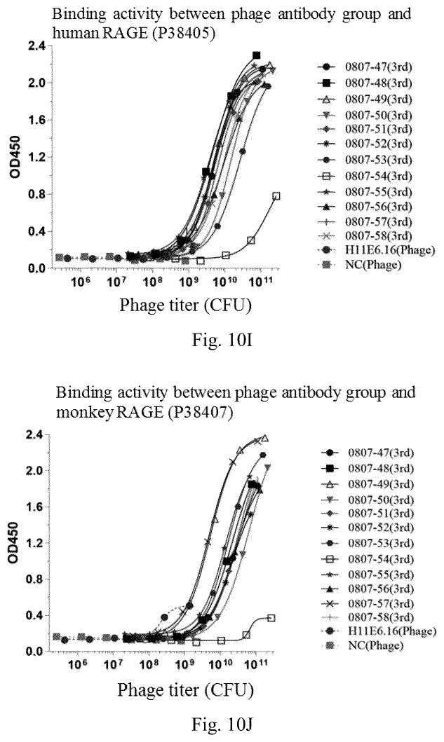

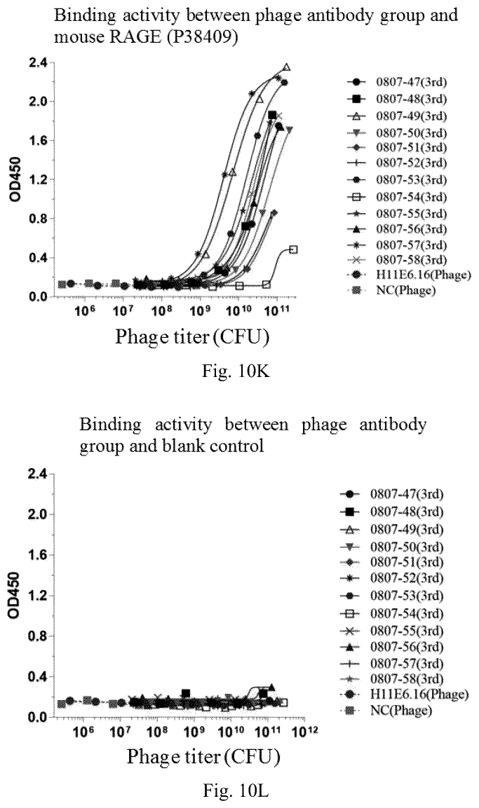

(5) TMB was added, the reaction was terminated with reaction termination solution, and OD450 was read. The experimental results are shown in FIGS. 10A-10L.

The experimental results show that phage antibody pools 0802-43, 0802-44, 0802-45, 0802-47, 0802-48, 0802-49, 0802-51, 0802-52, 0802-53, 0802-55, 0802-56, 0802-57, 0807-43, 0807-44, 0807-45, 0807-46, 0807-47, 0807-48, 0807-49, 0807-50, 0807-51, 0807-52, 0807-53, 0807-54, 0807-55, 0807-56, 0807-57, and 0807-58 have the characteristics of cross-enrichment among human, mouse and monkey, and the other pools have not the characteristics of cross-enrichment among human, mouse and monkey.

### Example 3: Identification of Positive Monoclonal Antibodies

### 3.1 Identifying Monoclonal Antibodies and Screening Positive Monoclonal Antibodies for Sequencing

### Experimental Method:

(1) The phage antibody-positive bacteria screened in Section 2.3 of Example 2 were sequenced.

(2) Each of the monoclonal antibodies in step (1) was subjected to Elisa validation (for the method, see Section 2.3 of Example 2), and the experimental results are summarized in the following Table 12:

TABLE 12

| | | | | | | hu-mu- | hu-mu- | | | | | |
|---|---|---|---|---|---|---|---|---|---|---|---|---|
| | | hu | hu | hu-cyno | hu-cyno | cyno | cyno | | | | | |
| | | Cross | Cross | Cross | Cross | Cross | Cross | Number of | | | | |
| | Selected | positive | positive | positive | positive | positive | positive | clones for | Analyzable | Unique | Unique | Final |
| Pool | clones | clones | rate | clones | rate | clones | rate | sequencing | sequences | number | rate | Unique |
| 0802-43(2nd) | 22 | 15 | 68.2% | 0 | 0.0% | 0 | 0.0% | 1 | 1 | 1 | 100.0% | 1 |
| 0802-44(2nd) | 20 | 15 | 75.0% | 3 | 15.0% | 2 | 10.0% | 3 | 3 | 2 | 66.7% | 2 |
| 0802-45(2nd) | 22 | 4 | 18.2% | 3 | 13.6% | 2 | 9.1% | 3 | 2 | 2 | 100.0% | 2 |
| 0802-47(2nd) | 16 | 11 | 68.8% | 3 | 18.8% | 2 | 12.5% | 3 | 3 | 3 | 100.0% | 3 |
| 0802-48(2nd) | 22 | 20 | 90.9% | 4 | 18.2% | 1 | 4.5% | 4 | 4 | 4 | 100.0% | 4 |
| 0802-49(2nd) | 20 | 7 | 35.0% | 5 | 25.0% | 2 | 10.0% | 5 | 4 | 4 | 100.0% | 4 |
| 0802-51(2nd) | 22 | 7 | 31.8% | 1 | 4.5% | 0 | 0.0% | 1 | 1 | 1 | 100.0% | 1 |
| 0802-52(2nd) | 16 | 16 | 100.0% | 6 | 37.5% | 1 | 6.3% | 6 | 6 | 5 | 83.3% | 5 |
| 0802-55(2nd) | 22 | 14 | 63.6% | 5 | 22.7% | 2 | 9.1% | 5 | 4 | 2 | 50.0% | 2 |
| 0802-56(2nd) | 20 | 8 | 40.0% | 0 | 0.0% | 0 | 0.0% | 0 | 0 | 0 | 0.0% | 0 |
| 0807-43(3rd) | 44 | 28 | 63.6% | 10 | 22.7% | 4 | 9.1% | 10 | 9 | 5 | 55.6% | 5 |
| 0807-44(3rd) | 40 | 36 | 90.0% | 18 | 45.0% | 7 | 17.5% | 18 | 9 | 8 | 88.9% | 8 |
| 0807-45(3rd) | 44 | 35 | 79.5% | 35 | 79.5% | 34 | 77.3% | 35 | 25 | 18 | 72.0% | 12 |
| 0807-47(3rd) | 40 | 37 | 92.5% | 10 | 25.0% | 6 | 15.0% | 10 | 3 | 3 | 100.0% | 2 |
| 0807-48(3rd) | 44 | 42 | 95.5% | 10 | 22.7% | 4 | 9.1% | 10 | 6 | 6 | 100.0% | 5 |
| 0807-49(3rd) | 40 | 39 | 97.5% | 39 | 97.5% | 39 | 97.5% | 39 | 25 | 16 | 64.0% | 13 |
| 0807-50(3rd) | 44 | 30 | 68.2% | 12 | 27.3% | 5 | 11.4% | 12 | 10 | 9 | 90.0% | 8 |
| 0807-51(3rd) | 22 | 18 | 81.8% | 8 | 36.4% | 2 | 9.1% | 8 | 5 | 5 | 100.0% | 4 |
| 0807-52(3rd) | 16 | 15 | 93.8% | 7 | 43.8% | 2 | 12.5% | 7 | 4 | 3 | 75.0% | 3 |
| 0807-53(3rd) | 40 | 24 | 60.0% | 24 | 60.0% | 20 | 50.0% | 24 | 19 | 11 | 57.9% | 8 |
| 0807-55(3rd) | 44 | 40 | 90.9% | 17 | 38.6% | 8 | 18.2% | 17 | 8 | 6 | 75.0% | 4 |
| 0807-56(3rd) | 40 | 30 | 75.0% | 6 | 15.0% | 4 | 10.0% | 6 | 2 | 2 | 100.0% | 2 |
| 0807-57(3rd) | 44 | 33 | 75.0% | 32 | 72.7% | 32 | 72.7% | 32 | 26 | 13 | 50.0% | 12 |
| 0807-58(3rd) | 40 | 18 | 45.0% | 5 | 12.5% | 5 | 12.5% | 5 | 4 | 3 | 75.0% | 2 |
| Statistics | 744 | 542 | 72.8% | 264 | 35.5% | 184 | 24.7% | 264 | 183 | 112 | 61.2% | 112 |

The experimental results show that among the 744 clones selected, 542 positive clones that bind to human antigens were screened, wherein 263 of them have cross-reactivity between human and monkey, and 184 of them have cross-reactivity among human, mouse and monkey. 264 positive clones were sent for sequencing, and 112 unique clones were obtained after sequence analysis, wherein 1 of them only binds to human antigens, 46 of them only have cross-reactivity between human and monkey, and 65 of them have cross-reactivity among human, mouse and monkey.

### Example 4: Activity Verification of Eukaryotically Expressed Antibodies and Antibody Fragments

4.1 Detection of the Binding Ability of Candidate Antibodies on p318 Cells

(1) Among the antibodies screened in Section 3.1 of Example 3, 31 candidate monoclonal antibodies with cross-reactivity among human, mouse and monkey were selected for eukaryotic expression.

(2) According to the screening strategy in Section 2.1 of Example 2, the p318-293F cells highly expressing RAGE antigens constructed in Section 1.7 of Example 1 and 293F cells were used for respectively plating at 1E5 cells/well, and the cells were washed with FACS buffer.

(3) 100 μL of antibody dilution was added to the cells to incubate at 4° C. for 60 min, then centrifuging, and washing the cells twice with FACS buffer.

(4) 100 μL of anti-human Fc (PE) (abcam ab98596 1:300) was added to the cells to incubate at 4° C. for 30 min.

(5) The cells were resuspended in FACS buffer, and detected by flow cytometry. The results of the mean fluorescence intensity of cells which show the binding ability of each candidate antibody on p318-293F cells are shown in FIGS. 11A-11B; the results of the mean fluorescence intensity of cells showing the binding ability of each candidate antibody on 293F cells are shown in Table 13.

TABLE 13

| | Tested samples | | | | | | | | |
|---|---|---|---|---|---|---|---|---|---|
| | NL-A9- | NL-A10- | NL-A69- | NL-A71- | NL-A72- | NL-A77- | NL-A78- | NL-A90- | NL-A96- |
| | VH(P57674) | VH(P57675) | VH(P57676) | VH(P57677) | VH(P57678) | VH(P57679) | VH(P57680) | VH(P57681) | VH(P57682) |
| MFI | 471.8 | 356.6 | 943.5 | 548.9 | 387.3 | 409.5 | 371 | 383.2 | 366 |
| | Tested samples | | | | | | | | |
| | NL-A104- | NL-A130- | NL-A134- | NL-A137- | NL-A138- | NL-A142- | NL-A144- | NL-A148- | NL-A154- |
| | VH(P57683) | VH(P57684) | VH(P57685) | VH(P57686) | VH(P57687) | VH(P57688) | VH(P57689) | VH(P57690) | VH(P57691) |
| MFI | 388.9 | 382.7 | 402.8 | 420.6 | 411.2 | 413.7 | 372.1 | 379.5 | 386.9 |
| | Tested samples | | | | | | | | |
| | NL-A162- | NL-A185- | NL-A190- | NL-A191- | NL-A201- | NL-A204- | NL-A223- | NL-A230- | NL-A247- |
| | VH(P57692) | VH(P57693) | VH(P57694) | VH(P57695) | VH(P57696) | VH(P57697) | VH(P57698) | VH(P57699) | VH(P57700) |
| MFI | 397.5 | 2382.8 | 394.5 | 2473.4 | 437.3 | 1066.9 | 581.9 | 420.3 | 372.2 |

| | Tested samples | | | | | | | |
|---|---|---|---|---|---|---|---|---|
| | NL-A254- | NL-A256- | NL-A257- | NL-A259- | | Anti- | | Cell |
| | VH(P57702) | VH(P57703) | VH(P57704) | VH(P57705) | h11E6.16(P38411) | hRAGE(P46085) | IgG1(P02183) | only |
| MFI | 458 | 1572.2 | 390.9 | 866.4 | 333.8 | 353.4 | 647.8 | 330.1 |

The experimental results show that, samples can bind to p318-293F cells except for NL-A69-VH (P57676), NL-A190-VH (P57694).

4.2 Detection of the Binding Activities of Eukaryotically Expressed Antibody Supernatant with Antigens Human-RAGE-ECD-Fc (P38406), Cyno-RAGE-ECD-Fc (P38408), and Mouse-RAGE-ECD-Fc (P38410)

(1) Among the antibodies screened in Section 3.1 of Example 3, 31 candidate monoclonal antibodies with cross-reactivity among human, mouse and monkey were selected for eukaryotic expression.

(2) Elisa plates were coated with different antigens human-RAGE-ECD-Fc (P38406), cyno-RAGE-ECD-Fc (P38408), and mouse-RAGE-ECD-Fc (P38410), wherein the antigens were diluted with PBS to a working concentration of 2 μg/mL, and 30 μL was added to each well to incubate at 4° C. overnight.

(3) The plates were washed three times with PBST, and then blocked with 5% skim milk (prepared with PBS) at room temperature for 2 h.

(4) The plates were washed three times with PBST, then adding phage antibody pool at a volume of 30 μL per well to incubate at room temperature for 60 min.

(5) The plates were washed three times with PBST, then adding goat-anti-mouse-IgG-Fc-HRP (abcam; ab97265) (diluted at 1:8000) or goat-anti-human-κ+λ-HRP (Millipore/AP502P, AP506P) (diluted at 1:4000) diluted with 1% skim milk to incubate at room temperature for 50 min.

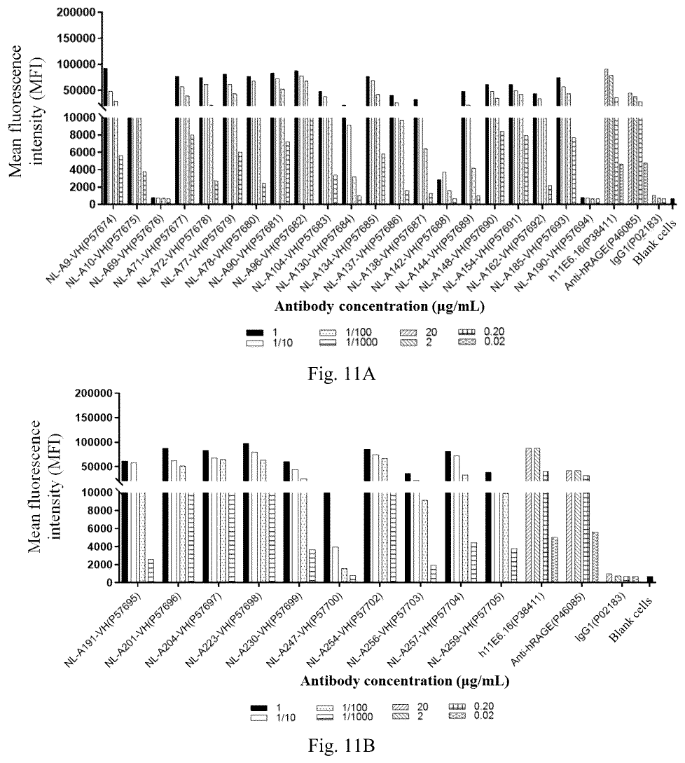

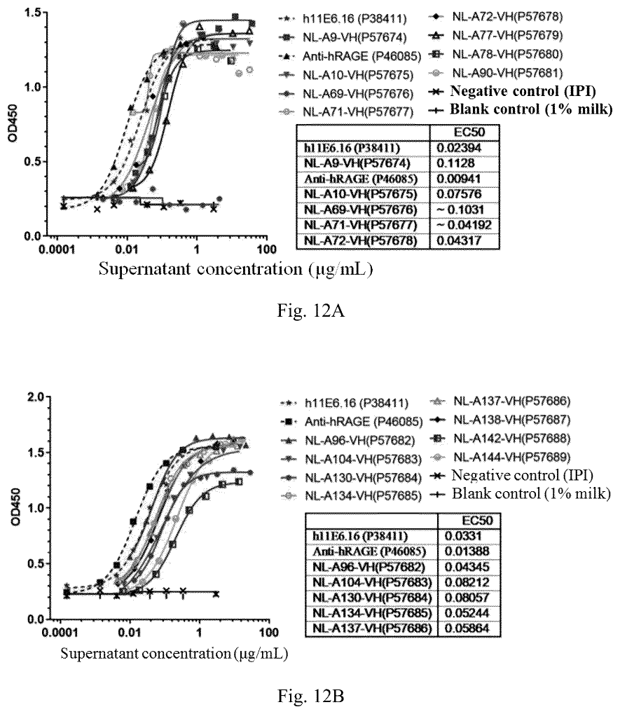

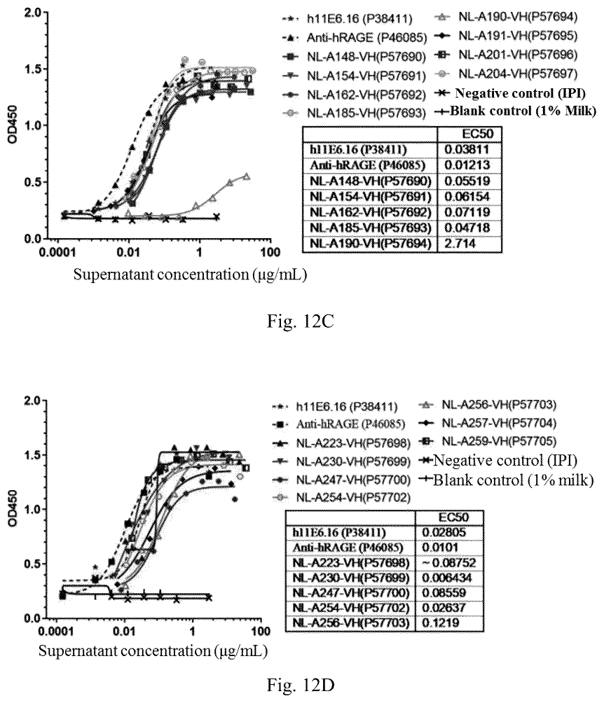

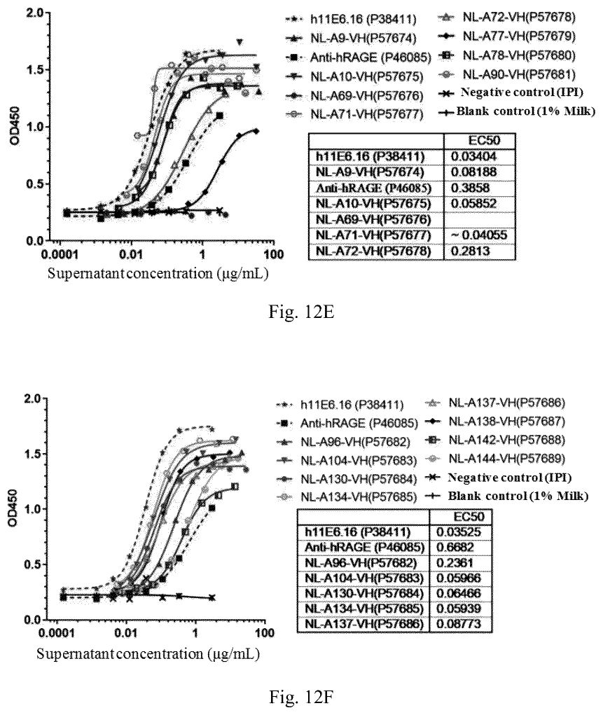

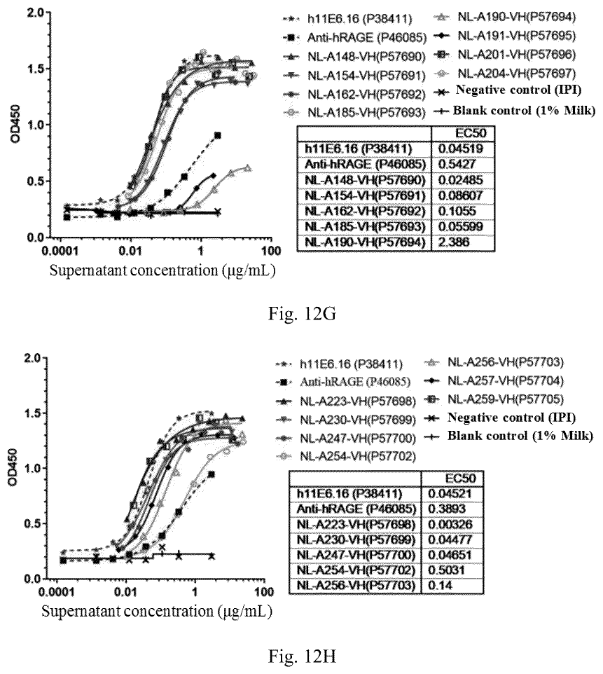

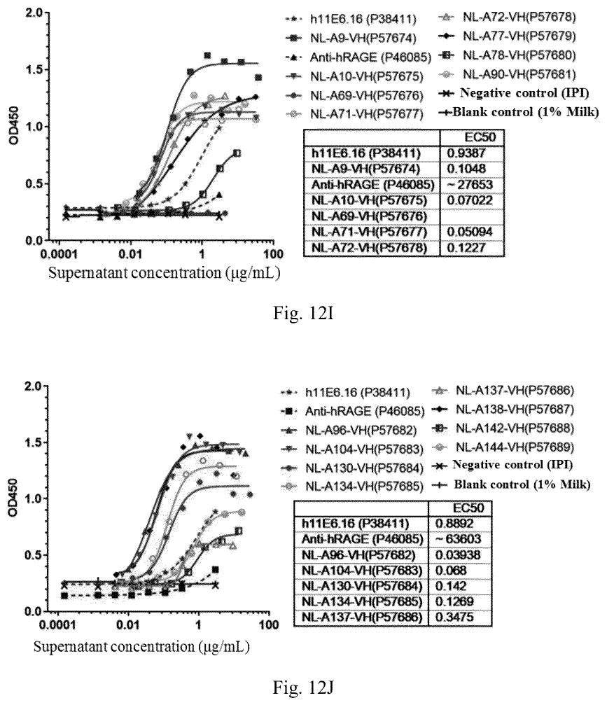

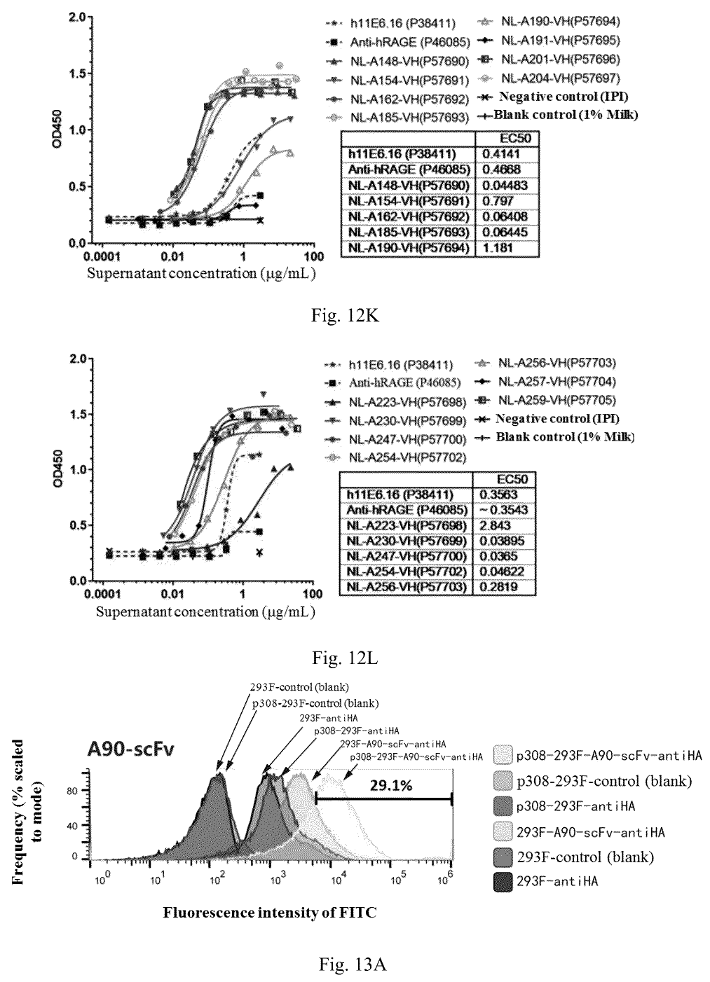

(6) TMB was added, the reaction was terminated with termination solution, and OD450 was read. The binding activity analysis results of the eukaryotically expressed antibody supernatant with human-RAGE-ECD-Fc (P38406) are shown in FIGS. 12A-12D, the binding activity analysis results of the eukaryotically expressed antibody supernatant with cyno-RAGE-ECD-Fc (P38408) are shown in FIGS. 12E-12H, and the binding activity analysis results of the eukaryotically expressed antibody supernatant with mouse-RAGE-ECD-Fc (P38410) are shown in FIGS. 12I-12L.

The experimental results show that, all 31 candidate antibodies screened by eukaryotic expression have cross-reactivity among human, mouse and monkey.

4.3 the antiRAGE scFv Antibody can Efficiently Bind to the Cells Expressing p318-293F Antigens

### Experimental Method:

(1) 6×His-antiRAGE(A90)-scFv-G4S-HA was expressed and synthesized, and the His-tagged protein was purified by using a nickel column; the designed configuration of the antiRAGE(A90)-scFv antibody fragment in each Example of the present application was VH-G4S-G4S-G4S-VL, wherein the VH sequence is represented by SEQ ID NO: 7, and the VL sequence is represented by SEQ ID NO: 8.

(2) The p318-293F cells constructed in Section 1.7 of Example 1 and 293F cells (negative control) were used, and the cells were washed and resuspended with PBS.

(3) The cells were fixed with 4% PFA for 20 min at room temperature, and then washed and resuspended with PBS.

(4) The cells were blocked with 5% BSA for 30 min at room temperature.

(5) 5 μg of antiRAGE(A90)-scFv was added to 100 μL of the incubation system to incubate at 4° C. for 2 h, and the cells were washed and resuspended with 0.5% BSA diluted in PBS.

(6) 1 μL of FITC-conjugated antiHA antibody was respectively added to cells without scFv antibody and cells incubated with scFv antibody to incubate at room temperature for 1 h. The cells were washed twice with PBS, and then resuspended with PBS.

(7) Flow cytometry was used for detecting the cells, and the experimental results are shown in FIG. 13A.

The experimental results show that, compared with the group without scFv antibody and the 293F cell group, antiRAGE(A90)-scFv antibody has a stronger binding affinity to p318-293F cells.

4.4 Anti-RAGE-Fc Antibody can Specifically Bind to HepG2 Cells Transiently Transfected with p605

### Experimental Method:

(1) Synthesis and expression: 6×His-antiRAGE(A90VH)-G4S-HA was paired with antiRAGE(A90VL) for expression, and His-tagged protein was purified by using a nickel column to obtain antiRAGE(A90)-Fc-G4S-HA; in each Example of the present application, the antiRAGE-Fc configuration is antiRAGE-Fc-G4S-HA-ALFA which is expressed by pairing the heavy chain with the light chain, wherein the configuration of antiRAGE(A90VH) is VH-CH1-CH2-CH3-G4S-HA-ALFA tag, and its amino acid sequence is represented by SEQ ID NO: 23 (QVQLVQSGAEVKKPGSSVKVSCKASGGTFNNYAINWARQAPGQGLEWMGRIVPFFDV TNYAQKFQGRVTITADKSTSTAYMELSSLRSEDTAIYYCAIGSFNWNSGAFDIWGQGTP VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYT LPPSRDELTKNQVSLTCLVKGFYPSDIAVEWESNGQPENNYKTTPPVLDSDGSFFLYSKL TVDKSRWQQGNVFSCSVMHEALHNHYTQKSLSLSPGKGGGGSYPYDVPDYAGGGGSP SRLEEELRRRLTE); the configuration of antiRAGE (A90VL) is VL-CL (Kappa), the amino acid sequence of VL is represented by SEQ ID NO: 8, and the sequence of CL (Kappa) is represented by SEQ ID NO: 10.

(2) The p605-HepG2 cells constructed in Section 1.8 of Example 1 were used, and the cells were washed and resuspended with PBS.

(3) The cells were fixed with 4% PFA for 20 min at room temperature, and then washed and resuspended with PBS.

(4) The cells were blocked with 5% BSA for 30 min at room temperature.

(5) 1 μg and 5 μg of antiRAGE(A90)-Fc were respectively added to 100 μL of the incubation system to incubate at 4° C. for 2 h. The cells were washed and resuspended with 0.5% BSA diluted in PBS.

(6) 1 μL of FITC-conjugated antiHA antibody was respectively added to cells without A90-Fc antibody and cells incubated with A90-Fc antibody to incubate at room temperature for 1 h. The cells were washed twice with PBS, and then resuspended with PBS.

(7) Flow cytometry was used for detection, and the experimental results are shown in FIG. 13B.

The experimental results show that, compared with the group without A90-Fc antibody, antiRAGE(A90)-Fc has a stronger binding affinity to p605 cells.

### Example 5: Display of RAGE Antibody Fragments on the Surface of Extracellular Vesicles

5.1 Display of antiRAGE scFv on EV Surface by Plasmid Transfection

### Experimental Method:

(1) Plasmids p641 (antiRAGE(A90)-G4S-flag-G4S-MFGE8(70-387)) and p642 (antiRAGE(A90)-G4S-flag-G4S-PLXNA1(863-1316)) were constructed, and MFGE8 truncation (positions 70-387 of the original sequence) and PLXNA1 truncation (positions 863-1316 of the original sequence) were used as EV scaffold proteins to fuse antiRAGE(A90)-scFv, respectively.

(2) EV preparation: p641 and p642 plasmids were transiently transfected into 293F cells, the expressing cells were cultured, and the cell supernatant was harvested to extract EVs. Untransfected wild-type 293 EVs were used as negative controls.

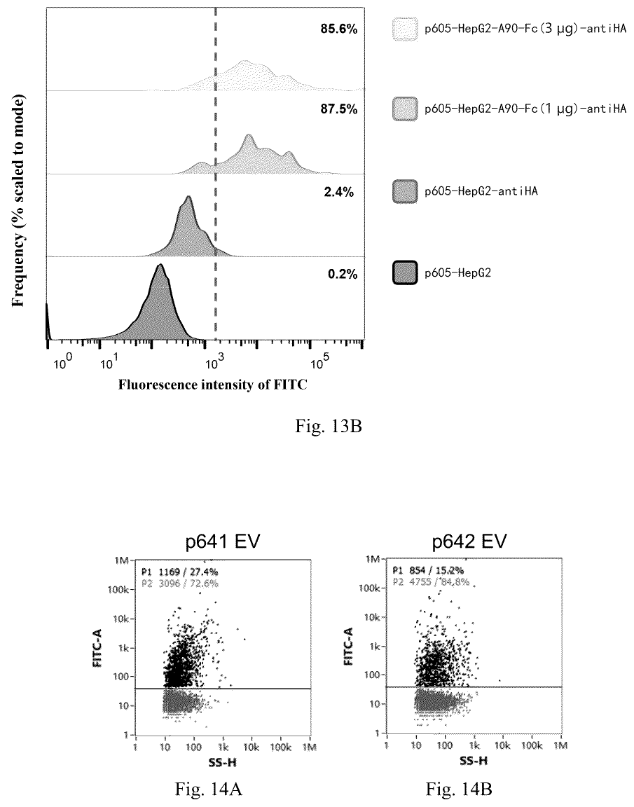

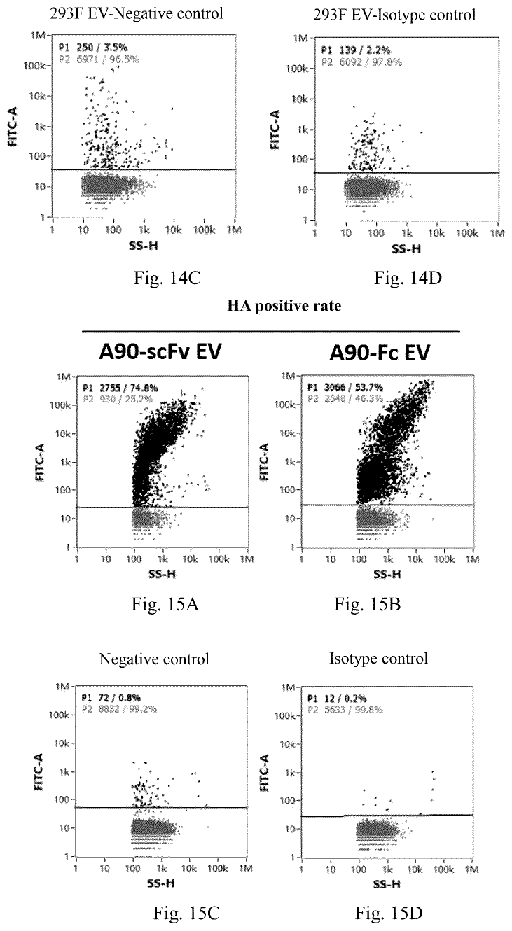

(3) Wild-type 293F EV, p641 EV, and p642 EV were labeled with FITC-conjugated anti-flag antibody (Proteintech/FITC-66008), and 293F EV was labeled with FITC-conjugated isotype control antibody (Proteintech/FITC-65128). The fluorescence intensity of the flag antibody was detected by nanoflow cytometry. The experimental results are shown in FIGS. 14A-14D.

The experimental results show that, based on the scaffold protein, antiRAGE(A90)-scFv can be displayed on the surface of EVs by constructing plasmids and transfecting donor cells.

5.2 Display of antiRAGE Antibody Fragments on the Surface of NbALFA EVs by Using Recombinant antiRAGE-scFv or antiRAGE-Fc Fused with ALFA Tag

### Experimental Method:

(1) NbALFA EVs were constructed by using the PLXNA1 truncation (positions 863-1316 of the original sequence) as a scaffold protein, and the EV particle concentration was detected by nanoflow cytometry.

(2) 6×His-antiRAGE(A90)-scFv-G4S-HA-G4S-ALFA was expressed and synthesized, and the His-tagged protein was purified by using a nickel column.

(3) 6×His-antiRAGE(A90VH)-G4S-HA-G4S-ALFA was paired with antiRAGE(A90VL) for expression, and the His-tagged protein was purified by using a nickel column to obtain antiRAGE(A90)-Fc-G4S-HA-G4S-ALFA.

(4) In each 100 μL PBS reaction system, 1E+9 NbALFA vesicles, 1% EV-free serum (FBS) and antiRAGE(A90)-scFv-HA-ALFA or antiRAGE(A90)-Fc-HA-ALFA protein molecules (the ratio of molecules to EV particles was 1000:1) were added to mix thoroughly and incubate at 25° C. for 30 min, and excess molecules were removed by size exclusion chromatography.

(5) The two EVs in step (4) were labeled with FITC-conjugated anti-HA antibodies, and the mean fluorescence intensity of HA was detected by nanoflow cytometry. The experimental results are shown in FIGS. 15A-15D.

The experimental results show that, antiRAGE-scFv and antiRAGE-Fc can be labeled on the EV surface with high loading capacity.

### Example 6

6.1 Validation of Targeting of antiRAGE EV to p605-HepG2 Cells

### Experimental Method:

(1) A90-Fc EV was constructed by the method in Section 5.2 of Example 5. NbALFA EV without antibody fragment labeling was used as control EV. EV was stained with DSPE-PEG-FITC dye, and EV particle concentration was detected by nanoflow cytometry.

(2) The p605-HepG2 cells constructed by the method in Section 1.8 of Example 1 were used as antigen-expressing cells, and the untransfected HepG2 cells were used as negative cells.

(3) p605-HepG2 cells and HepG2 cells were fixed with 4% PFA at room temperature for 20 min, then washing and resuspending the cells with PBS.

(4) p605-HepG2 cells and HepG2 cells were blocked with 5% BSA at room temperature for 30 min, then washing and resuspending the cells with PBS.

(5) FITC-labeled A90-Fc EVs and control EVs were co-incubated with p605-HepG2 cells and HepG2 cells, respectively. The ratio of the number of EV particles to cells was respectively set to 10,000, 15,000, 20,000, 25,000 and 30,000 particles/cell. The cells were incubated at 4° C. with low-frequency shaking for 2 h, then washing with PBS to remove excess extracellular vesicles.

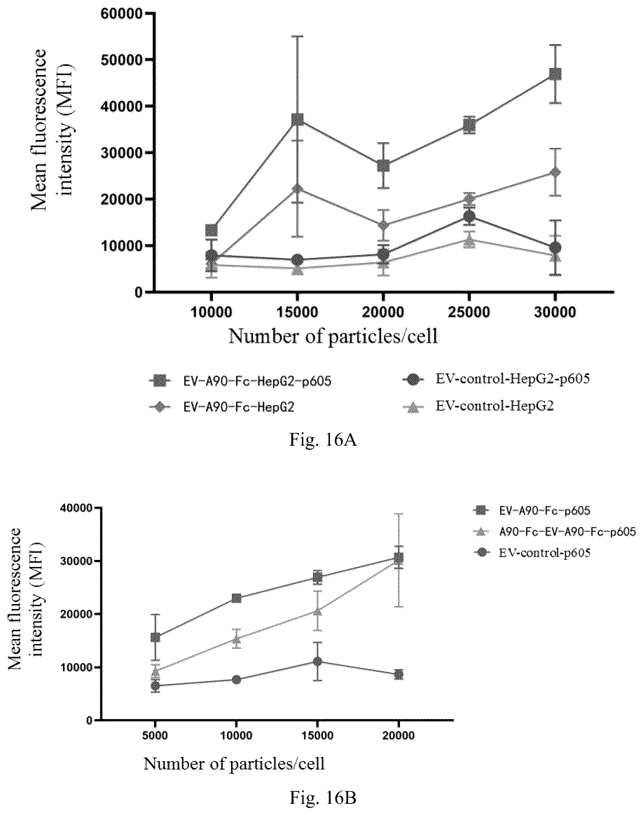

(6) The FITC fluorescence intensity was detected by flow cytometer, and the experimental results are shown in FIG. 16A.

The experimental results show that, compared with the negative cell group and the control EV group, A90-Fc EV has a stronger affinity for the antigen-expressing cell p605-HepG2.

6.2 Validation of Specific Targeting of antiRAGE EV to p605-HepG2 Cells Experimental Method:

(1) A90-Fc EVs were constructed by the method in Section 5.2 of Example 5. NbALFA EVs without antibody fragment labeling were used as control EVs. EVs were stained with DSPE-PEG-FITC dye, and the EV particle concentration was detected by nanoflow cytometry.

(2) The p605 plasmid was transiently transfected into HepG2 cells as antigen-expressing cells.

(3) p605-HepG2 cells were fixed with 4% PFA at room temperature for 20 min, then washing and resuspending the cells with PBS.

(4) p605-HepG2 cells were blocked with 5% BSA at room temperature for 30 min, then washing and resuspending the cells with PBS.

(5) FITC-labeled A90-Fc EVs and control EVs were co-incubated with p605-HepG2 cells, respectively, and the ratio of the number of EV particles to cells was respectively set to 5000, 10000, 15000 and 20000 particles/cell. The cells were incubated at 4° C. with low-frequency shaking for 2 h, and then washing with PBS to remove excess vesicles. At the same time, a group of p605-HepG2 cells were first incubated with 0.5 μg of A90-Fc recombinant protein at room temperature for 30 min, then adding A90-Fc EV labeled with FITC dye. The subsequent experimental conditions were the same.

(6) The FITC fluorescence intensity was detected by flow cytometer. The experimental results are shown in FIG. 16B.

The experimental results show that, the addition of A90-Fc recombinant protein first has a blocking effect on the binding between A90-Fc EVs and p605-HepG2 cells, indicating that the binding between A90-Fc EVs and p605-HepG2 cells depends on the affinity of A90-Fc for RAGE antigen; and when A90-Fc EV is excessive (feed ratio 20000 particles/cell), the blocking effect of A90-Fc recombinant protein is weakened.

The above is only description of preferred embodiments of the present application, and does not constitute any other form of limitation to the present application. Any technician familiar with the present profession may use the technical contents disclosed above to change or modify them into equivalent embodiments with equivalent changes. However, any simple modifications, equivalent changes and modifications made to the above embodiments based on the technical essence of the present application without departing from the content of the technical solutions of the present application still fall within the protection scope of the technical solutions of the present application.

1. An anti-RAGE antibody, wherein the antibody comprises three heavy chain complementary determining regions (CDR-H) and three light chain complementary determining regions (CDR-L), the three heavy chain complementary determining regions are CDR-H1, CDR-H2 and CDR-H3, and the three light chain complementary determining regions are CDR-L1, CDR-L2 and CDR-L3, wherein
the CDR-H1 comprises the amino acid sequence represented by SEQ ID NO: 1;
the CDR-H2 comprises the amino acid sequence represented by SEQ ID NO: 2;
the CDR-H3 comprises the amino acid sequence represented by SEQ ID NO: 3;
the CDR-L1 comprises the amino acid sequence represented by SEQ ID NO: 4;
the CDR-L2 comprises the amino acid sequence represented by SEQ ID NO: 5; and
the CDR-L3 comprises the amino acid sequence represented by SEQ ID NO:6.

2. The anti-RAGE antibody according to claim 1, comprising a human universal framework region.

3. The anti-RAGE antibody according to claim 1, wherein the antibody comprises a heavy chain variable region and a light chain variable region, wherein
the heavy chain variable region comprises the amino acid sequence represented by SEQ ID NO:7, or an amino acid sequence having at least 90%, 91%, 92%, 93%, 94%, 95%, 96%, 97%, 98% or 99% identity with SEQ ID NO:7; and
the light chain variable region comprises the amino acid sequence represented by SEQ ID NO:8, or an amino acid sequence having at least 90%, 91%, 92%, 93%, 94%, 95%, 96%, 97%, 98%, or 99% identity with SEQ ID NO:8.

4. The anti-RAGE antibody according to claim 1, which is a human antibody, a humanized antibody or a chimeric antibody.

5. The anti-RAGE antibody according to claim 1, which is a full-length IgG antibody.

6. The anti-RAGE antibody according to claim 1, which is a full-length IgG1 antibody.

7. The anti-RAGE antibody according to claim 1, which is any one antigen-binding fragment selected from the group consisting of: Fv, Fab, F(ab′)2, Fab′, Fd, dsFv, scFv, sc(Fv)2, dAb, isolated complementary determining region, domain-specific antibody, single domain antibody, domain-deleted antibody, CDR-grafted antibody, diabody, triabody, tetrabody and minibody.

8. The anti-RAGE antibody according to claim 1, which binds to RAGE antigens derived from human, monkey and murine.

9. A conjugate comprising the anti-RAGE antibody according to claim 1.

10. A bispecific or multispecific antibody comprising the anti-RAGE antibody according to claim 1.

11. An immune cell comprising the anti-RAGE antibody according to claim 1 or a nucleic acid encoding such an antibody.

12. An antibody modified with lipophilic group, wherein the antibody is the anti-RAGE antibody according to claim 1.

13. A fusion protein comprising the anti-RAGE antibody according to claim 1 and one or more polypeptides or proteins.

14. A nucleic acid molecule, encoding the anti-RAGE antibody according to claim 1.

15. An expression vector, comprising the nucleic acid molecule according to claim 14.

16. A cell, comprising the nucleic acid molecule according to claim 14.

17. An extracellular vesicle, virus, liposome, cell, organelle or non-biological material, displaying the anti-RAGE antibody according to claim 1.

18. A method for displaying the anti-RAGE antibody according to claim 1 on extracellular vesicles, comprising:
performing fusion expression of a scaffold protein or a truncation or variant thereof with the anti-RAGE antibody according to claim 1 on the membrane of extracellular vesicles; or
connecting the anti-RAGE antibody according to claim 1 to the membrane of extracellular vesicle via an affinity pairing molecule; or
connecting the anti-RAGE antibody according to claim 1 to the membrane of extracellular vesicle by click chemistry reagents or by covalent bonding; or
embedding the anti-RAGE antibody according to claim 1 in the membrane of extracellular vesicle by lipophilic group.

19. A pharmaceutical composition, comprising the extracellular vesicle, virus, liposome, cell, organelle or non-biological material according to claim 17, and a pharmaceutically acceptable carrier.

20. A method for diagnosing, treating or preventing a disease, comprising administering to a subject in need thereof an effective amount of the extracellular vesicle, virus, liposome, cell, organelle or non-biological material according to claim 17.


SEQUENCE LISTING (23 sequences)

SEQ ID NO: 1 - protein, 6 aa, synthetic construct
NNYAIN

SEQ ID NO: 2 - protein, 10 aa, synthetic construct
RIVPFFDVTN

SEQ ID NO: 3 - protein, 12 aa, synthetic construct
GSFNWNSGAFDI

SEQ ID NO: 4 - protein, 8 aa, synthetic construct
QSISSWLA

SEQ ID NO: 5 - protein, 9 aa, synthetic construct
IYQASSLES

SEQ ID NO: 6 - protein, 8 aa, synthetic construct
QYKDYPLT

SEQ ID NO: 7 - protein, 121 aa, synthetic construct
QVQLVQSGAEVKKPGSSVKVSCKASGGTFNNYAINWARQAPGQGLEWMGRIVPFFDVTNYAQKFQGRVTITADKSTSTAYMELSSLRSEDTAIYYCAIGSFNWNSGAFDIWGQGTPVTVSS

SEQ ID NO: 8 - protein, 107 aa, synthetic construct
NIQLTQSPSTLSASVGDRVTITCRASQSISSWLAWYQQKPGKAPKLLIYQASSLESGVPSRFSGSGSGTEFTLTIRSLQPEDFAVYFCQQYKDYPLTFGGGTKVDIK

SEQ ID NO: 9 - protein, 324 aa, synthetic construct
ASTKGPSVFPLAPSSKSTSGGTAALGCLVKDYFPEPVTVSWNSGALTSGVHTFVLQSSGLYSLSSVVTVPSSSLGTQTYICNVNHKPSNTKVDKKVEPKSCDKTHTCPPCPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - DNA, 1509 bp, synthetic construct
  source 1..1509: mol_type = other DNA
atgggttggagcctcatcttgctcttccttgtcgctgttgctacgcgtgtccactcccaggttcagctggtgcagtctggggctgaggtgaagaagcctgggtcctcggtgaaggtctcctgcaaggcttctggaggcaccttcaataactatgctatcaactgggcgcgacaggcccctggacaagggcttgagtggatgggaaggatcgtccctttctttgatgtgacaaactacgcacagaaattccagggcagggtcacgattaccgcggacaaatccacgagcacagcctacatggaattgagcagcctgagatctgaggacacggccatatattactgtgcgatagggtcgtttaactggaactcgggtgcttttgatatctggggccaggggaccccggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaaggaggcggaggctcttacccttacgatgtgcctgattacgctggcggaggcggcagcccttctagactggaagaagaactgcggcggagactgaccgaa

SEQ ID NO: 12 - DNA, 699 bp, synthetic construct
  source 1..699: mol_type = other DNA
atgggttggagcctcatcttgctcttccttgtcgctgttgctacgcgtgtccactccaacatccagttgacccagtctccttccaccctgtctgcatctgtaggcgacagagtcaccatcacttgccgggccagtcagagtattagtagctggttggcctggtatcagcagaaaccagggaaagcccctaaactcctgatctatcaggcgtctagtttagaaagtggggtcccatcaaggttcagcggcagtggatctgggacagaattcactctcaccatccgcagcctacagcctgaagattttgcagtttatttctgccaacagtataaagattaccctctcactttcggcggagggaccaaggtggatatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 13 - protein, 552 aa, synthetic construct
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 551 aa, synthetic construct
AQNITARIGEPLVLKCKGAPKKPPQQLEWKLNTGRTEAWKVLSPQGGPWDSVARVLPNGSLFLPAVGIQDEGIFRCQAMNRNGKETKSNYRVRVYQIPGKPEIIDSASELTAGVPNKVGTCVSEGSYPAGTLSWHLDGKPLVPNEKGVSVKEETRRHPETGLFTLQSELMVTPARGGNPHPTFSCSFSPGLPRRRALHTAPIQPRVWEPVPLEEVQLVVEPEGGAVAPGGTVTLTCEVPAQPSPQIHWMKDGVPLPLSPSPVLILPEIGPQDQGTYRCVATHPSHGPQESRAVSISIIEPGEEGPTAGSVGGSGPGTLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 550 aa, synthetic construct
GQNITARIGEPLVLSCKGAPKKPPQQLEWKLNTGRTEAWKVLSPQGGPWDSVARILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHEDEGTYSCVATHPSHGPQESPPVSIRVTETGDEGPAEGSVGESGLGTLAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 334 aa, synthetic construct
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLAGGSGGSHHHHHHHH

SEQ ID NO: 17 - protein, 333 aa, synthetic construct
AQNITARIGEPLVLKCKGAPKKPPQQLEWKLNTGRTEAWKVLSPQGGPWDSVARVLPNGSLFLPAVGIQDEGIFRCQAMNRNGKETKSNYRVRVYQIPGKPEIIDSASELTAGVPNKVGTCVSEGSYPAGTLSWHLDGKPLVPNEKGVSVKEETRRHPETGLFTLQSELMVTPARGGNPHPTFSCSFSPGLPRRRALHTAPIQPRVWEPVPLEEVQLVVEPEGGAVAPGGTVTLTCEVPAQPSPQIHWMKDGVPLPLSPSPVLILPEIGPQDQGTYRCVATHPSHGPQESRAVSISIIEPGEEGPTAGSVGGSGPGTLAGGSGGSHHHHHHHH

SEQ ID NO: 18 - protein, 332 aa, synthetic construct
GQNITARIGEPLVLSCKGAPKKPPQQLEWKLNTGRTEAWKVLSPQGGPWDSVARILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHEDEGTYSCVATHPSHGPQESPPVSIRVTETGDEGPAEGSVGESGLGTLAGGSGGSHHHHHHHH

SEQ ID NO: 19 - protein, 450 aa, synthetic construct
EIQLVQSGAEVKKPGASVKVSCKASGYTFTNFGMNWVRQAPGQGLEWMGYINTNTGESIYSEEFKGRFTFTLDTSTSTAYMELSSLRSEDTAVYFCARSRMVTAYGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
EIVMTQSPATLSLSPGERATLSCKASQNVGTAVAWYQQKPGQSPRLLIFSASNRYTGVPARFSGSGSGTDFTLTISSLQSEDFAVYFCQQYSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 662 aa, synthetic construct
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGPDYKDHDGDYKDHDIDYKDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 22 - protein, 665 aa, synthetic construct
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGPDYKDHDGDYKDHDIDYKDDDDKMVSKGEELFTGVVPILVELDGDVNGHKFSVSGEGEGDATYGKLTLKFICTTGKLPVPWPTLVTTLSHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGSVQLADHYQQNTPIGDGPVLLPDSHYLSTQSALSKDPNEKRDHMVLLEFVTAAGITLGMDELYK

SEQ ID NO: 23 - protein, 484 aa, synthetic construct
QVQLVQSGAEVKKPGSSVKVSCKASGGTFNNYAINWARQAPGQGLEWMGRIVPFFDVTNYAQKFQGRVTITADKSTSTAYMELSSLRSEDTAIYYCAIGSFNWNSGAFDIWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSYPYDVPDYAGGGGSPSRLEEELRRRLTE